SEQUENCE LISTING (5 sequences)

SEQ ID NO: 1 - DNA, 26 bp, ARTIFICIAL SEQUENCE
  Primer sequence
agactctgcgtgctgctggcaaaaac

SEQ ID NO: 2 - DNA, 25 bp, ARTIFICIAL SEQUENCE
  Primer sequence
cgtggaaagacctttgtcccacctg

SEQ ID NO: 3 - DNA, 25 bp, ARTIFICIAL SEQUENCE
  Primer sequence
gggccagctcattcctcccactcat

SEQ ID NO: 4 - DNA, 66 bp, ARTIFICIAL SEQUENCE
  VK-1 signal peptide coding sequence
atggacatgagggtgcccgctcagctcctggggctcctgctgctgtggctgagaggtgcgcgctgt

SEQ ID NO: 5 - protein, 22 aa, ARTIFICIAL SEQUENCE
  Synthetic Construct
MDMRVPAQLLGLLLLWLRGARC

(19) United States
(12) Patent Application Publication
(10) Pub. No.: US 2013/0115171 A1
(43) Pub. Date: May 9, 2013

(54) NAV1.7-RELATED ASSAYS

(71) Applicant: Stefan I. MCDONOUGH, Cambridge, MA (US)

(72) Inventor: Stefan I. MCDONOUGH, Cambridge, MA (US)

(21) Appl. No.: 13/353,202

(22) Filed: Jan. 18, 2012

Related U.S. Application Data
(60) Provisional application No. 61/557,877, filed on Nov. 9, 2011.

(51) Int. Cl. A61K 49/00

(52) U.S. Cl. 424/9.2

(57) ABSTRACT

A viable global Na_{V}1.7^{-/-} knockout mouse is disclosed, and a breeding colony of global Na_{V}1.7^{-/-} knockout mice. Also disclosed are an isolated mouse gamete that does not encode a functional Na_{V}1.7, produced by the Na_{V}1.7^{-/-} knockout mouse; an isolated Na_{V}1.7^{-/-} mouse cell, or a progeny cell thereof, isolated from the Na_{V}1.7^{-/-} knockout mouse; and a primary cell culture or a secondary cell line and a tissue or organ explant or culture thereof derived from the Na_{V}1.7^{-/-} knockout mouse. Disclosed also are a hybridoma, wherein the hybridoma was originally formed from the fusion of the isolated Na_{V}1.7^{-/-} mouse cell mouse cell and a myeloma cell, and a method of making an antibody. Also disclosed are assays useful for screening prospective Na_{V}1.7 inhibitors and dose ranging a test Na_{V}1.7 inhibitor compound, which were validated using the Na_{V}1.7^{-/-} knockout mouse.

This application claims the benefit of U.S. Provisional Application No. 61/557,877, filed Nov. 9, 2011, which is hereby incorporated by reference in its entirety.

### FIELD OF THE INVENTION

The instant application contains an ASCII “txt” compliant sequence listing which serves as both the computer readable form (CRF) and the paper copy required by 37 C.F.R. Section 1.821(c) and 1.821(e), and is hereby incorporated by reference in its entirety. The name of the “txt” file created on Jan. 17, 2012, is: A-1588-US-NP2-SegList011812.5T25.txt, and is 2 kb in size.

Throughout this application various publications are referenced within parentheses or brackets. The disclosures of these publications in their entireties are hereby incorporated by reference in this application in order to more fully describe the state of the art to which this invention pertains.

### BACKGROUND OF THE INVENTION

1. Field of the Invention

The present invention relates to the field of drug screening assays.

2. Discussion of the Related Art

While the use of isolated cell lines (i.e., an in vitro system) is helpful in understanding the physiological role of various genes and the proteins they give rise to, more complete information can be obtained by studying the effects role of these proteins directly in a mammal (i.e., an in vivo system). To this end, various mammals have been produced that have altered levels of expression of certain genes. One class of these mammals is so-called transgenic mammals. These mammals latter have a novel gene or genes, originating from a different species, introduced into their intact genome, hence the “transgenic” qualification. Another class is the knock-in mammals. These animals have one of their own genes deleted and replaced by a variant of that same gene. This approach is often used to produce a hyper or hypomorph of the gene/protein of choice. Kinases and proteins with functional phosphorylated site(s) are the targets of choice for this approach. A combination of the first two techniques can be used to create a “transgenic-knock-in” mammal that expresses a foreign gene in the locus of the endogenous host gene; such as a human gene in the mouse locus of the equivalent gene. The final approach is to create a global null mutant, or so-called “knockout” mammals, wherein expression of an endogenous gene has been suppressed through genetic manipulation, whether by using recombinant or classical genetic techniques. For example, Nassar et al. (2006) generated a global null mutant mouse line unable to express the voltage-gated sodium channel Na_{V} 1.3. (Nassar et al., Nerve injury induces robust allodynia and ectopic discharges in Nay 1.3 null mutant mice, Mol. Pain. 2:33 (2006)).

Voltage-gated sodium channels (VGSC) are glycoprotein complexes responsible for initiation and propagation of action potentials in excitable cells such as central and peripheral neurons, cardiac and skeletal muscle myocytes, and neuroendocrine cells. Mammalian sodium channels are heterotrimers, composed of a central, pore-forming alpha (α) subunit and auxiliary beta (β) subunits. Mutations in alpha subunit genes have been linked to paroxysmal disorders such as epilepsy, long QT syndrome, and hyperkalemic periodic paralysis in humans, and motor endplate disease and cerebellar ataxia in mice. (Isom, Sodium channel beta subunits: anything but auxiliary, Neuroscientist 7(1):42-54 (2001)). The β-subunit modulates the localization, expression and functional properties of α-subunits in VGSCs.

Voltage gated sodium channels comprise a family consisting of 9 different subtypes (Na_{V}1.1-Na_{V}1.9). As shown in Table 1, these subtypes show tissue specific localization and functional differences (See, Goldin, A. L., Resurgence of sodium channel research, Annu Rev Physiol 63: 871-94 (2001); Wilson et al., Compositions useful as inhibitors of voltage-gated ion channels, US 2005/0187217 A1). Three members of the gene family (Na_{V}1.8, 1.9, 1.5) are resistant to block by the well-known sodium channel blocker tetrodotoxin (TTX), demonstrating subtype specificity within this gene family. Mutational analysis has identified glutamate 387 as a critical residue for TTX binding (See, Noda, M., H. Suzuki, et al., A single point mutation confers tetrodotoxin and saxitoxin insensitivity on the sodium channel II″ FEBS Lett 259(1): 213-6 (1989)).

TABLE 1
VGSC family with rat TTX IC50 values.

| | | TTX | |
|---|---|---|---|
| VGSC | | IC50 | |
| isoform | Tissue | (nM) | Indication |
| Na_{V}1.1 | CNS, PNS | 10 | Pain, Epilepsy, |
| | soma of | | Neurodegeneration |
| | neurons | | |
| Na_{V}1.2 | CNS | 10 | Neurodegeneration, Epilepsy |
| | high in axons | | |
| Na_{V}1.3 | CNS, | 2-15 | Pain, Epilepsy |
| | embryonic, | | |
| | injured nerves | | |
| Na_{V}1.4 | Skeletal muscle | 5 | Myotonia |
| Na_{V}1.5 | Heart | 2000 | Arrhythmia, long QT |
| Na_{V}1.6 | CNS | 1 | Pain, movement disorders |
| | widespread, most | | |
| | abundant | | |
| Na_{V}1.7 | PNS, DRG, | 4 | Pain, Neuroendocrine disorders, |
| | terminals | | prostate cancer |
| | neuroendocrine | | |
| Na_{V}1.8 | PNS, small neurons | >50,000 | Pain |
| | in DRG & TG | | |
| Na_{V}1.9 | PNS, small neurons | 1000 | Pain |
| | in DRG & TG | | |

Abbreviations:
CNS = central nervous system,
PNS = peripheral nervous system,
DRG = dorsal root ganglion,
TG = Trigeminal ganglion.
(See, Wilson et al., Compositions useful as inhibitors of Voltage-gated ion channels, US 2005/0187217 A1; Goldin, Resurgence of Sodium Channel Research, Annu Rev Physiol 63: 871-94 (2001)).

In general, voltage-gated sodium channels (Nays) are responsible for initiating the rapid upstroke of action potentials in excitable tissue in nervous system, which transmit the electrical signals that compose and encode normal and aberrant pain sensations. Antagonists of Na_{V} channels can attenuate these pain signals and are useful for treating a variety of pain conditions, including but not limited to acute, chronic, inflammatory, and neuropathic pain. Known Na_{V} antagonists, such as TTX, lidocaine, bupivacaine, phenyloin, lamotrigine, and carbamazepine, have been shown to be useful for attenuating pain in humans and animal models. (See, Mao, J. and L. L. Chen, Systemic lidocaine for neuropathic pain relief, Pain 87(1): 7-17 (2000); Jensen, T. S., Anticonvulsants in neuropathic pain: rationale and clinical evidence, Eur J Pain 6 (Suppl A): 61-68 (2002); Rozen, T. D., Antiepileptic drugs in the management of cluster headache and trigeminal neuralgia, Headache 41 Suppl 1: S25-32 (2001); Backonja, M. M., Use of anticonvulsants for treatment of neuropathic pain, Neurology 59(5 Suppl 2): S14-7 (2002)).

The α-subunits of TTX-sensitive Na_{V}1.7 channels are encoded by the SCN9A gene. The Na_{V}1.7 channels are preferentially expressed in peripheral sensory neurons of the dorsal root ganglia, which are involved in the perception of pain. In humans, mutations in the SCN9A gene have been associated with predispositions to pain hyper- or hypo-sensitivity. For instance, a role for the Na_{V}1.7 channel in pain perception was established by recent clinical gene-linkage analyses that revealed gain-of-function mutations in the SCN9A gene as the etiological basis of inherited pain syndromes such as primary erythermalgia (PE), inherited erythromelalgia (IEM), and paroxysmal extreme pain disorder (PEPD). (See, e.g., Yang et al., Mutations in SCN9A, encoding a sodium channel alpha subunit, in patients with primary erythermalgia, J. Med. Genet. 41:171-174 (2004); Harty et al., Na_{V}1.7 mutant A863P in erythromelalgia: effects of altered activation and steady-state inactivation on excitability of nociceptive dorsal root ganglion neurons, J. Neurosci. 26(48):12566-75 (2006); Estacion et al., Na_{V}1.7 gain-of-function mutations as a continuum: A1632E displays physiological changes associated with erythromelalgia and paroxysmal extreme pain disorder mutations and produces symptoms of both disorders, J. Neurosci. 28(43):11079-88 (2008)). In addition, overexpression of Na_{V}1.7 has been detected in strongly metastatic prostate cancer cell lines. (Diss et al., A potential novel marker for human prostate cancer: voltage-gated sodium channel expression in vivo, Prostate Cancer and Prostatic Diseases 8:266-73 (2005); Uysal-Onganer et al., Epidermal growth factor potentiates in vitro metastatic behavior human prostate cancer PC-3M cells: involvement of voltage-gated sodium channel, Molec. Cancer 6:76 (2007).

Loss-of-function mutations of the SCN9A gene result in a complete inability of an otherwise healthy individual to sense any form of pain. (e.g., Ahmad et al., A stop codon mutation in SCN9A causes lack of pain sensation, Hum. Mol. Genet. 16(17):2114-21 (2007)).

A cell-specific deletion of the SCN9A gene in conditional knockout mice reduces their ability to perceive mechanical, thermal or inflammatory pain. (Nassar et al., Nociceptor-specific gene deletion reveals a major role for Na_{V}1.7 (PN1) in acute and inflammatory pain, Proc. Natl. Acad. Sci, USA. 101(34): 12706-12711 (2004)).

Based on such evidence, decreasing Na_{V}1.7 channel activity or expression levels in peripheral sensory neurons of the dorsal root ganglia has been proposed as an effective pain treatment, e.g. for chronic pain, neuropathic pain, and neuralgia. (E.g., Thakker et al., Suppression of SCN9A gene expression and/or function for the treatment of pain, WO 2009/033027 A2; Yeomans et al., Decrease in inflammatory hyperalgesia by herpes vector-mediated knockdown of Na_{V}1.7 sodium channels in primary afferents, Hum. Gene Ther. 16(2):271-7 (2005); Fraser et al., Potent and selective Na_{V}1.7 sodium channel blockers, WO 2007/109324 A2; Hoyt et al., Discovery of a novel class of benzazepinone Na(v)1.7 blockers: potential treatments for neuropathic pain, Bioorg. Med. Chem. Lett. 17(16):4630-34 (2007); Hoyt et al., Benzazepinone Na_{V}1.7 blockers: Potential treatments for neuropathic pain, Bioorg. Med. Chem. Lett. 17(22):6172-77 (2007)).

Nassar et al. used gene ablation in mice to examine the function of Na_{V}1.7 in pain pathways; however, they reported that global Na_{V}1.7-null mutants were found (unlike humans) to die shortly after birth, apparently because of a failure to feed. (Nassar et al., Nociceptor-specific gene deletion reveals a major role for Na_{V}1.7 (PN1) in acute and inflammatory pain, Proc Natl Acad Sci USA. 101(34): 12706-12711 (2004)). Indeed, of the 92 pups that survived, 72% were heterozygotes and the rest were Na_{V}1.7 wild types. Nassar et al. (2004) stated that “ . . . deleting Na_{V}1.7 in all sensory and sympathetic neurons causes a lethal perinatal phenotype.” (Nassar et al., ibid., at page 12708). In view of the neonatal lethality that Nassar et al. observed, they used a Cre-loxP approach to generate nociceptor-specific knockouts. These tissue restricted KO were described as animals that no longer express Nav1.7 in a subset of sensory and sympathetic neurons, but express Nav1.7 everywhere else in the body. The mice were generated by crossing Na_{V}1.8 Cre-deletor mice with floxed Na_{V}1.7 mice to generate tissue-restricted Na_{V}1.7^{-/-} mice and littermate controls. These nociceptor-specific animals were then used to study mechanisms in nociception and pain.

Nassar et al. stated in a separate report that “[i]t is not possible to generate global knockouts of both Na_{V}1.8 and Na_{V}1.7 since global deletion of Na_{V}1.7 is lethal at P0.” (Nassar et al., Neuropathic pain develops normally in mice lacking both Na_{V}1.7 and Na_{V}1.8, Mol. Pain. 1-24 (2005)).

In contrast to the art mentioned above, the present invention provides, inter alia, global Na_{V}1.7 null mutant mice and fertile Na_{V}1.7 knockout mouse lines for the study of Na_{V}1.7-mediated physiology and for the development of pharmaceuticals, for example, particularly targeting pain and neuroendocrine disorders.

### SUMMARY OF THE INVENTION

Within the present invention, an assay, involving a Na_{V}1.7-specific biochemical challenge, was validated using the global Na_{V}1.7^{-/-} knockout mice. The assay is useful, for example, for screening prospective Na_{V}1.7 inhibitors, which may be useful for research or clinical purposes. The assay, comprises:

(a) dosing a mammal (e.g., a mouse, rat, rabbit, ferret, dog, non-human primate, or human) with a test compound (a candidate Na_{V}1.7 inhibitor), followed by

(b) dosing the mammal with a dose of a Na_{V}1.7 activator (e.g., veratridine, deltamethrin, or grayanotoxin III) effective to induce a pain-associated response in a negative control (not receiving the test compound); such dosing can be systemic or local; and then

(c) determining whether the pain-associated response in the mammal is reduced compared to the negative control. As mentioned, test compound administration to the mammal can be systemic (e.g., by intraperitoneal, intravenous, intramuscular, or oral administration) or local (e.g., by subcutaneous, intraplanar, or topical administration). If local, it should be at or near the same location as a local dose of the Nav1.7 activator.

In another embodiment of the invention, sodium channel activators are used in an assay, involving a Na_{V}1.7-specific biochemical challenge, useful for choosing local or systemic doses of Na_{V}1.7-blocking test compounds. Selecting the proper dose of any test compound in clinical trials is a difficult task, ideally done by calibrating the dose to one that displaces a biomarker of some sort, e.g., a PET ligand, known to be specific for the target. Sodium channels in general and Na_{V}1.7 in particular have heretofore had no such biomarker. Herein we disclose that activators of sodium channels, including veratridine, deltamethrin, and grayanotoxin, produce a quantifiable behavioral response when injected into the paw of rats or mice at proper doses. These three molecules are structurally different, but share a common physiological mechanism in that each activates Na_{V}1.7. In Example 5, we show for the first time that rats and mice each display quantifiable, dose-dependent flinching and licking behavior upon injection of any of these sodium channel activators. These behaviors are reduced by morphine and prevented by the nonselective sodium channel antagonist mexiletine, verifying that the behaviors reflect pain and are mediated by sodium channels.

Most significantly, we show in Example 5 herein that a Na_{V}1.7 activator, e.g., veratridine dosed at 1 microgram, produces no flinching or licking behavior when injected into the paw of global Nav1.7−/− mice, whereas this same 1 microgram dose produces a robust flinching behavior in wild type mice. Pharmacological block of Na_{V}1.7 should achieve the same effect, since pre-administration to wild type mice of mexiletine (which blocks Na_{V}1.7 as well as all other sodium channels) prevents the flinching behavior evoked by the 1-microgram dose of veratridine. Accordingly, in preclinical studies, challenge with a sodium channel activator is a useful test of whether a given compound administered to a living animal is blocking Na_{V}1.7. Furthermore, this test could be used clinically to determine proper dosing of a test Na_{V}1.7 inhibitor to treat a clinical pain syndrome. A proper clinical dose would be one that prevents a painful response to administration of a sodium channel activator.

In one embodiment, the present invention includes an assay, useful for dose ranging a test compound (a candidate Na_{V}1.7 inhibitor), comprising:

(a) dosing a first mammal (e.g., a mouse, rat, rabbit, ferret, dog, non-human primate, or human) at a first dose of a test compound, followed by

(b) dosing the first mammal with a (local or systemic) dose of a Na_{V}1.7 activator (e.g., veratridine, deltamethrin, or grayanotoxin III) effective to induce a pain-associated response in a negative control (not receiving the test compound); such dosing can be systemic or local; and then

(c) determining whether the pain-associated response is reduced in the first mammal compared to the negative control; and

(d) identifying a lowest second dose of the test compound at which the pain-associated response is reduced compared to the negative control. Test compound administration to the mammal can be systemic (e.g., by intraperitoneal, intravenous, intramuscular, or oral administration) or local (e.g., by subcutaneous, intraplanar, or topical administration). If local, it should be at or near the same location as a local dose of Na_{V}1.7 activator. The first mammal can be re-used at a later time, for dosing at a second dose level different from the first, after a recovery period sufficient such that the effects test compound and Na_{V}1.7 activator are worn-off and the presence of any residual test compound and Na_{V}1.7 activator compound in the mammal is undetectable. Alternatively, the assay can further include dosing a second mammal of the same species at the second dose of the test compound, followed by dosing the second mammal with the dose of the Na_{V}1.7 activator effective to induce the pain-associated response in a negative control; and then determining whether the pain-associated response is reduced in the second mammal compared to the negative control.

In another embodiment, the present invention includes an assay, useful for dose ranging a test compound (a candidate Na_{V}1.7 inhibitor), comprising:

(a) dosing a first mammal (e.g., a mouse, rat, rabbit, ferret, dog, non-human primate, or human) at a first dose of a test compound and a second mammal (of the same species) at a second dose of the test compound different from the first dose, followed by

(b) dosing the first and the second mammals with a dose of a Na_{V}1.7 activator (e.g., veratridine, deltamethrin, or grayanotoxin III) effective to induce a pain-associated response in a negative control (not receiving the test compound); such dosing can be systemic or local; and then

(c) determining whether the pain-associated response is reduced in the first mammal and the second mammal compared to the negative control; and

(d) identifying a lowest second dose of the test compound at which the pain-associated response is reduced compared to the negative control. Test compound administration to the mammal can be systemic (e.g., by intraperitoneal, intravenous, intramuscular, or oral administration) or local (e.g., by subcutaneous, intraplanar, or topical administration). If local, it should be at or near the same location as a local dose of Nav1.7 activator.

In this manner the dose to inhibit Na_{V}1.7 can be determined and compared to presumably higher doses that may give adverse effects, to determine therapeutic window. Moreover, in clinical trials of efficacy of a test Na_{V}1.7 inhibitor, only at such doses can therapeutic efficacy be ascribed to Na_{V}1.7. The key knowledge, provided by the global Na_{V}1.7−/− mice, is that sodium channel activators produce a painful response via Na_{V}1.7 and only Na_{V}1.7.

The present invention is also directed to a viable global Na_{V}1.7^{-/-} knockout mouse, in surprising contrast to teachings in the art that a global Na_{V}1.7 knockout mutation is lethal in mice as early as in the post-natal day 0 (P0) generation. (E.g., Nassar et al., Nociceptor-specific gene deletion reveals a major role for Na_{V}1.7 (PN1) in acute and inflammatory pain, Proc Natl Acad Sci USA. 101(34): 12706-12711 (2004); Nassar et al., Neuropathic pain develops normally in mice lacking both Na_{V}1.7 and Na_{V}1.8, Mol. Pain. 1-24 (2005)). By carefully observing the lack of vigor exhibited by newborn Nav1.7^{-/-} mice in the same C57BL/6J background employed by Nassar et al., and deliberately choosing different strains with enhanced vigor relative to C57BL/6J, and wherein the females displayed enhanced maternal nurturing behavior relative to C57BL/6J, we have been able to produce such viable global Na_{V}1.7^{-/-} knockout mice derived from these more vigorous strain backgrounds.

In one embodiment of the invention, the mouse is an outcrossed or backcrossed global Na_{V}1.7^{-/-} knockout mouse, or a progeny mouse derived therefrom that is also Na_{V}1.7^{-/-}. The global Na_{V}1.7^{-/-} knockout mouse or its Na_{V}1.7^{-/-} progeny can also be mated with Na_{V}1.7^{+/+} partners of the same strain or a different strain to produce other progeny with a genotype that is Na_{V}1.7^{+/-}; the global Na_{V}1.7^{-/-} knockout mouse or its Na_{V}1.7^{-/-} progeny can also be mated with Na_{V}1.7^{+/-} partners of the same strain or a different strain to produce other progeny with a genotype that is Na_{V}1.7^{+/-} or Na_{V}1.7^{-/-}.

In another embodiment of the invention, the global Na_{V}1.7^{-/-} knockout mouse is an adult.

In another embodiment of the invention, a Na_{V}1.7^{-/-} mouse cell (e.g., a B-lymphocyte, T cell, or neuronal cell), can be isolated from the global Na_{V}1.7^{-/-} knockout mouse, and progeny cells, a primary cell culture or a secondary cell line are thus derived from the global Na_{V}1.7^{-/-} knockout mouse.

In other embodiments of the invention, tissue or organ explants, or cultures thereof, are also derived from the global Na_{V}1.7^{-/-} knockout mouse.

Since the inventive global Na_{V}1.7^{-/-} knockout mouse adult includes fertile male and female individuals, another aspect of the present invention relates to a breeding colony of global Na_{V}1.7^{-/-} knockout mice, comprising at least one breeding pair of adult global Na_{V}1.7^{-/-} knockout mice.

In another embodiment of the invention, a hybridoma can be made by fusion of the Na_{V}1.7^{-/-} mouse B-lymphocyte cell, mentioned above, and a myeloma cell.

In another aspect of the invention, preparation of antibodies against Na_{V}1.7, including but not limited to, murine or human Na_{V}1.7. Based on the CDR sequences of the anti-human Na_{V}1.7 antibodies produced by the inventive Na_{V}1.7^{-/-} knockout mice, chimeric or humanized antibodies can be developed incorporating those CDRs into an antibody for either antagonizing or agonizing Na_{V}1.7 ion channel activity, which can be of therapeutic value.

In other embodiments, the inventive global Na_{V}1.7^{-/-} knockout mice are useful for drug research and development, for example, in in vivo protocols to distinguish on-target/off-target effects or distinguish between pain and sedation effects.

Numerous additional aspects and advantages of the present invention will become apparent upon consideration of the figures and detailed description of the invention.

### BRIEF DESCRIPTION OF THE DRAWINGS

FIG. 1A-B illustrates schematically the prior art concept of backcrossing (FIG. 1A) and outcrossing (FIG. 1B) breeding strategies. Backcrossing consists of completely changing the background of an inbred mouse line into another inbred background. (In FIG. 1A designated C57BL/6 or 129SV strains are merely illustrative.) When ˜99.99% of the new genetic background of the inbred line of choice (e.g., C57BL/6) is obtained, the new mouse line is considered congenic. It can occur at the initial stage of creating a knockout line (as shown), or at any later time point if the initial inbred mouse line is no longer suitable for the current research. In such a case, one starts with a 99.99% mouse of the initial line and breed until one obtains ˜99.99% of the newly selected inbred line. In FIG. 1B, outcrossing involves only one cross with an outbred mouse line (e.g., CD1). A hybrid mouse line is obtained with ˜50% of the genes of the initial inbred line and ˜50% of the genes of the outbred line (e.g., CD1).

FIG. 2A-B shows that outcrossing B6.129P2-Scn9^{atm1Dgen}/J animals to a CD1 or backcrossing to a BALB/c background (similar data not shown) increased the survival of the animals up to 7 days postnatally, allowing us to investigate feeding behavior. FIG. 2A shows a one-week-old Na_{V}1.7^{-/-} neonate (black) with control littermates (white) obtained from an outcross using the CD1 mouse line. This Na_{V}1.7^{-/-} animal reached adulthood. FIG. 2B shows a newborn Na_{V}1.7 control (left) and Na_{V}1.7^{-/-} (right); both were capable of feeding on their own as seen by the presence of milk in their stomachs (indicated by arrows). (The skin is quasi-transparent in neonatal mice.)

FIG. 3A-G shows structural development of the central and peripheral nervous system appears normal in Na_{V}1.7^{-/-} animals. FIG. 3A shows a sagittal section of a hematoxylin and eosin stained Na_{V}1.7^{-/-} neonate (postnatal day 4 (P4)). Hematoxylin and eosin (H&E) are standard histology markers used in pathology to assess integrity of the tissue. Both the nuclei (darker spots; blue in original) and the rest of the cell (lighter regions; original in various shades of pink) are labeled by H&E. FIG. 3B-F show magnification of various regions of the central nervous system and FIG. 3G shows a magnification of a region of the peripheral nervous system: Cortex (FIG. 3B); Hippocampus (FIG. 3C); Cerebellum (FIG. 3D; see arrow); olfactory bulb (FIG. 3E); spinal cord (FIG. 3F); and dorsal root ganglion (DRG; FIG. 3G). Magnification is about 20×.

FIG. 4A-E shows that development of the internal organs appears normal in Na_{V}1.7^{-/-} animals. FIG. 4A shows a sagittal section of a Na_{V}1.7^{-/-} neonate (postnatal day 4 (P4)) head. Note the structural integrity of the nasal cavity and septum (upper arrow) as well as that of the tongue (lower arrow) and jaw. FIGS. 4B-E show magnifications of internal organs found in their respective and expected regions: lung (FIG. 4B); heart (FIG. 4C); kidney (FIG. 4D); small intestine (triple arrow points to lumen) and bladder (single arrow)(FIG. 4E). Magnification is about 20×.

FIG. 5A-B shows artificial mouse milk production. FIG. 5A shows a 4-liter batch of artificial mouse milk in preparation: FIG. 5B illustrates aliquots of the final product and feeding tool for the neonate mice; shown is a 25-μL glass Hamilton syringe combined with a 24 gauge feeding needle.

FIG. 6 shows hand feeding of a postnatal day-10 Na_{V}1.7^{-/-} candidate in overall good health and with a shiny coat held in a gloved human hand. Eyes of mouse pups are not typically opened at that age.

FIG. 7A-C illustrates a DNA electrophoresis gel used to determine the genotype of the mice in our Na_{V}1.7 colonies. AMA-161 was the first confirmed weaned Na_{V}1.7^{-/-} (KO) animal (on a CD1 background). For comparison, animal AMA-50 was a confirmed Na_{V}1.7^{+/+} (wild type) individual. Primer sequences were obtained from Deltagen (San Mateo, Calif.) and were used to genotype all animals in the colonies: Forward Scn9a primer: 5′-AGA CTC TGC GTG CTG CTG GCA AAA AC-3′(SEQ ID NO:1); Forward Neomycin primer: 5′-GGG CCA GCT CAT TCC TCC CAC TCA T-3′(SEQ ID NO:3); and Reverse Scn9a primer: 5′-CGT GGA AAG ACC TTT GTC CCA CCT G-3′ (SEQ ID NO:2). These primers gave rise to an endogenous (E) band of 267 base pairs primer (Forward Scn9a+primer Reverse Scn9a; see bands in FIG. 7A), or a targeted (T) band of 389 base pairs (primer Forward Neomycin+primer Reverse Scn9a; see bands in FIG. 7B). FIG. 7C shows controls of PCR reaction samples that did not contain DNA. Lane 1 is the control PCR for the endogenous product and Lane 2 is the control PCR for the targeted product; as expected neither lane gave rise to a PCR band. Reference Molecular Ladder lanes (L) are from a commercial source: TriDye™ 100 bp DNA ladder (New England BioLabs Inc., Ipswich, Mass.; catalog number N3271S). White arrows indicate the molecular size of the corresponding bands.

FIG. 8A-D illustrates the external phenotype of Na_{V}1.7 KO mice (see mice indicated by arrows in FIG. 8A-D). As early as 16 hours after birth, Na_{V}1.7^{-/-} animals were smaller than their littermates. The external phenotype of Na_{V}1.7^{-/-} pups was normal, except for the noticeable difference in size. Their eyes were open, their teeth erupted, and their coats were well developed. They were mobile in the cage approximately at the same time as their littermates, albeit with a few days of delay.

FIG. 8E shows a size comparison over the course of 8 weeks post-weaning. Animals (AMA-627 to −631) originated from the same SCn9a-CD1 litter.

FIG. 9A-B illustrates that Na_{V}1.7^{-/-} mice have a minimal pain response in a thermal challenge test (Hargreaves Apparatus). FIG. 9A shows results from Scn9a-CD1 Na_{V}1.7 KO mice, which exhibited a delayed pain response (right paw, n=3) or no reaction (left paw, n=5). FIG. 9B shows results for Scn9a-BalbC Na_{V}1.7 KO mouse (n=1), which did not respond in either paw to thermal challenge. No differences were seen between WT and HET in either FIG. 9A or FIG. 9B, i.e., all reacted normally.

FIG. 10A-H illustrates the response of Na_{V}1.7 KO mice to increasing thermal pain (i.e. hot plate test). Notably, Na_{V}1.7 KO mice (Scn9a-CD1, FIG. 10A-D; n=14; and Scn9a-BalbC, FIG. 10E-H; n=4) were insensitive to thermal pain, showing no response at all even at the highest testable temperature of 55° C. (FIG. 10D and FIG. 10H), at which they had to be removed at the cut off (20 seconds) to avoid severe superficial tissue damage.

FIG. 11A-B shows representative results from tactile allodynia-Von Frey test All Na_{V}1.7 KO mice (Scn9a-CD1, FIG. 11A; n=16; and Scn9a-BalbC, FIG. 11B; n=4) reacted normally to a Von Frey allodynia challenge. All reached cut-off threshold of 1.5 g. hence, Na_{V}1.7 KO animals appear to perceive mechanical pressure normally.

FIG. 12A-B shows representative results from anosmia testing. Na_{V}1.7 KO mice had difficulties (Scn9a-CD1, FIG. 12A; n=, 14), or failed (Scn9a-BalbC, FIG. 12B; n=4), in locating a hidden scented food pellet, compared to age-matched/sex-matched control (WT/HET) littermates.

FIG. 13A-B demonstrates that, unlike their WT/HET littermates, Na_{V}1.7 KO mice (Scn9a-CD1, FIG. 13A; n=12; and Scn9a-BalbC, FIG. 13B; n=3) were insensitive to histamine induced itch behavior. The average number of scratch bouts performed by the Na_{V}1.7 KO were similar to that of saline injection in wild type/heterozygous control littermates.

FIG. 14 shows in vitro modulation of Na_{V}1.7 by veratridine. Currents through hNa_{V}1.7 stably expressed in HEK 293 cells were evoked by a family of depolarizing voltage pulses at 10-mV intervals from a holding voltage of −100 mV, using the whole-cell configuration of the patch-clamp technique.

FIG. 15 shows paw lifting/licking (left panel) and flinching (right panel) behaviors in rats, induced by veratridine and the effect of the indicated doses of mexiletine thereon. * means p<0.05, ** means p<0.01, *** means p<0.001.

FIG. 16 shows total lifting time in male CD1 mice recorded for 30 minutes following intraplantar (i.pl.) injection of the indicated dose of veratridine in 1% ethanol in phosphate-buffered saline and the inhibition of the behavior by pre-administration of mexiletine. *** means p<0.001; ### means p<0.001 compared to a secondary group.

FIG. 17A-B shows total flinches in male CD1 mice in response to a 10-microgram suspension dose of deltamethrin (FIG. 17A; n=6) or a 0.1-microgram dose of grayanotoxin III (FIG. 17B; n=6) in solution with 1% ethanol in phosphate-buffered saline. The effect of pre-administration of mexiletine at 30 mg/kg i.p. in saline solution is also shown. * means p<0.05, ** means p<0.01.

FIG. 18 shows that veratridine injection produced a robust flinching response in wild type heterozygote CD1 mice, while the same amount and volume of veratridine produced no response in CD1 Na_{V}1.7 knockouts. One microgram of veratridine was injected into the paws of adult global knockout Na_{V}1.7 mice (n=5) and wild type heterozygote littermates (n=6). Mexiletine pre-administration to wildtype/heterozygote mice prevented flinching otherwise induced by veratridine. *** means p<0.001.

FIG. 19A-C show that deltamethrin and grayanotoxin III, which elicited a flinching response from rats, each activated Na_{V}1.7. Recordings shown were whole-cell patch-clamp electrophysiology records of human Na_{V}1.7 stably expressed in a HEK 293 cell line. FIG. 19A (control) shows overlaid currents in response to a series of test step depolarizations from −85 mV to +15 mV, in +5 mV increments. Holding voltage and repolarization voltage was −85 mV; currents shown are not leak-subtracted. FIG. 19B shows currents from the same cell after bath exposure to 1 micromolar deltamethrin. Voltage dependence of activation was unchanged, but note the incomplete inactivation during the step depolarization, and the extended inward currents, corresponding to sodium entry into the cell, upon repolarization. X-scalebars, one nanoampere; y-scalebars, twenty milliseconds. Currents shown in FIG. 19C are from a different cell expressing hNa_{V}1.7, with 300 micromolar grayanotoxin III in the internal (pipette) solution. Tested from a holding voltage of −120 mV (left), test step depolarizations from −120 mV to −50 mV (in +5 mV increments) activated sodium currents, starting at −95 mV, that did not inactivate during the test pulse. With holding voltage switched to −80 mV, the holding current grew larger (dashed line), reflecting continuous opening of Na_{V}1.7. Further step depolarizations from −80 mV to −40 mV evoked slowly-deactivating currents. Scalebars, 500 picoamps and 20 milliseconds.

FIG. 20A-B demonstrates antibody generation by Nav1.7 knockout mice.

### DETAILED DESCRIPTION OF EMBODIMENTS

The section headings used herein are for organizational purposes only and are not to be construed as limiting the subject matter described.

### DEFINITIONS

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a”, “an” and “the” include plural referents unless the context clearly indicates otherwise. For example, reference to “a protein” includes a plurality of proteins; reference to “a cell” includes populations of a plurality of cells.

“Polypeptide” and “protein” are used interchangeably herein and include a molecular chain of two or more amino acids linked covalently through peptide bonds. The terms do not refer to a specific length of the product. Thus, “peptides,” and “oligopeptides,” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide. The terms also include molecules in which one or more amino acid analogs or non-canonical or unnatural amino acids are included as can be expressed recombinantly using known protein engineering techniques. In addition, fusion proteins can be derivatized as described herein by well-known organic chemistry techniques.

The term “recombinant” indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well known molecular biological procedures. Examples of such molecular biological procedures are found in Maniatis et al., Molecular Cloning. A Laboratory Manual. Cold Spring Harbor Laboratory, Cold Spring Harbor, N.Y. (1982). A “recombinant DNA molecule,” is comprised of segments of DNA joined together by means of such molecular biological techniques. The term “recombinant protein” or “recombinant polypeptide” as used herein refers to a protein molecule which is expressed using a recombinant DNA molecule. A “recombinant host cell” is a cell that contains and/or expresses a recombinant nucleic acid.

The term “polynucleotide” or “nucleic acid” includes both single-stranded and double-stranded nucleotide polymers containing two or more nucleotide residues. The nucleotide residues comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phosphoraniladate and phosphoroamidate.

The term “oligonucleotide” means a polynucleotide comprising 200 or fewer nucleotide residues. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including an isotopic label (e.g., ¹²⁵I, ¹⁴C, ¹³C, ³⁵S, ³H, ²H, ¹³N, ¹⁵N, ¹⁸O, ¹⁷O etc.), for ease of quantification or detection, a fluorescent label, a hapten or an antigenic label, for detection assays. Oligonucleotides may be used, for example, as PCR primers, cloning primers or hybridization probes.

A “polynucleotide sequence” or “nucleotide sequence” or “nucleic acid sequence,” as used interchangeably herein, is the primary sequence of nucleotide residues in a polynucleotide, including of an oligonucleotide, a DNA, and RNA, a nucleic acid, or a character string representing the primary sequence of nucleotide residues, depending on context. From any specified polynucleotide sequence, either the given nucleic acid or the complementary polynucleotide sequence can be determined. Included are DNA or RNA of genomic or synthetic origin which may be single- or double-stranded, and represent the sense or antisense strand. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

As used herein, an “isolated nucleic acid molecule” or “isolated nucleic acid sequence” is a nucleic acid molecule that is either (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined. An isolated nucleic acid molecule is other than in the form or setting in which it is found in nature. However, an isolated nucleic acid molecule includes a nucleic acid molecule contained in cells that ordinarily express a polypeptide (e.g., an oligopeptide or antibody) where, for example, the nucleic acid molecule is in a chromosomal location different from that of natural cells.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of ribonucleotides along the mRNA chain, and also determines the order of amino acids along the polypeptide (protein) chain. The DNA sequence thus codes for the RNA sequence and for the amino acid sequence.

The term “gene” is used broadly to refer to any nucleic acid associated with a biological function. Genes typically include coding sequences and/or the regulatory sequences required for expression of such coding sequences. The term “gene” applies to a specific genomic or recombinant sequence, as well as to a cDNA or mRNA encoded by that sequence. A “fusion gene” contains a coding region that encodes a polypeptide with portions from different proteins that are not naturally found together, or not found naturally together in the same sequence as present in the encoded fusion protein (i.e., a chimeric protein). Genes also include non-expressed nucleic acid segments that, for example, form recognition sequences for other proteins. Non-expressed regulatory sequences including transcriptional control elements to which regulatory proteins, such as transcription factors, bind, resulting in transcription of adjacent or nearby sequences.

“Expression of a gene” or “expression of a nucleic acid” means transcription of DNA into RNA (optionally including modification of the RNA, e.g., splicing), translation of RNA into a polypeptide (possibly including subsequent post-translational modification of the polypeptide), or both transcription and translation, as indicated by the context.

As used herein the term “coding region” or “coding sequence” when used in reference to a structural gene refers to the nucleotide sequences which encode the amino acids found in the nascent polypeptide as a result of translation of an mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

The term “control sequence” or “control signal” refers to a polynucleotide sequence that can, in a particular host cell, affect the expression and processing of coding sequences to which it is ligated. The nature of such control sequences may depend upon the host organism. In particular embodiments, control sequences for prokaryotes may include a promoter, a ribosomal binding site, and a transcription termination sequence. Control sequences for eukaryotes may include promoters comprising one or a plurality of recognition sites for transcription factors, transcription enhancer sequences or elements, polyadenylation sites, and transcription termination sequences. Control sequences can include leader sequences and/or fusion partner sequences. Promoters and enhancers consist of short arrays of DNA that interact specifically with cellular proteins involved in transcription (Maniatis, et al., Science 236:1237 (1987)). Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see Voss, et al., Trends Biochem. Sci., 11:287 (1986) and Maniatis, et al., Science 236:1237 (1987)).

The term “vector” means any molecule or entity (e.g., nucleic acid, plasmid, bacteriophage or virus) used to transfer protein coding information into a host cell.

The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Such techniques are well known in the art. (E.g., Goodey, Andrew R.; et al., Peptide and DNA sequences, U.S. Pat. No. 5,302,697; Weiner et al., Compositions and methods for protein secretion, U.S. Pat. No. 6,022,952 and U.S. Pat. No. 6,335,178; Uemura et al., Protein expression vector and utilization thereof, U.S. Pat. No. 7,029,909; Ruben et al., 27 human secreted proteins, US 2003/0104400 A 1).

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. For example, a control sequence in a vector that is “operably linked” to a protein coding sequence is ligated thereto so that expression of the protein coding sequence is achieved under conditions compatible with the transcriptional activity of the control sequences.

The term “host cell” means a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial host cells in culture include bacteria (such as Escherichia coli sp.), yeast (such as Saccharomyces sp.) and other fungal cells, insect cells, plant cells, mammalian (including human) cells, e.g., CHO cells and HEK-293 cells. Modifications can be made at the DNA level, as well. The peptide-encoding DNA sequence may be changed to codons more compatible with the chosen host cell. For E. coli, optimized codons are known in the art. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA may recombine with that of the cell by physically integrating into a chromosome of the cell, or may be maintained transiently as an episomal element without being replicated, or may replicate independently as a plasmid. A cell is considered to have been “stably transformed” when the transforming DNA is replicated with the division of the cell.

The process of modifying an inbred mouse strain (e.g., C57Bl/6J) onto a different inbred mouse strain (e.g., BALB/c) is referred to as “backcrossing” or “backcross”. This is useful when a mouse strain is not ideal for the intended research purpose; the strain can be genetically modified through breeding schemes. To completely transform the genotype from “strain A” (e.g., C57Bl/6J) to “strain B” (e.g., BALB/c), mice typically need to be backcrossed at least 10 times; only then can they be referred to as “congenic”. To backcross mice, the mutant mice are bred with mice from the inbred strain of choice (e.g., BALB/c). Offspring of this breeding are termed: “backcross #1” (or “N1”) and are hybrids of both strains (e.g., approximately: 50% C57Bl6/J and 50% BALB/c). The offspring can be genotyped and only those heterozygous for the gene of interest will be bred again with a wild type individual of the inbred mouse strain (e.g., wild type BALB/c mice). Typically, this procedure is repeated about 10 times until a congenic BALB/c line is obtained. Any combinations of inbred mouse lines can be used in this fashion to create a congenic line of the strain of choice. As an example, by December 2010, we had generated and characterized the fifth backcross generation (“N5”) onto the inbred BALB/c background; the heterozygous (“HET”) offspring are ˜98.6% BALB/c in genetic background. In May of 2011, we obtained our first BALB/c congenic breeding pairs. We are currently mating them to assess whether this backgrounds improves the viability of Na_{V}1.7 KO neonates (i.e requiring less human care/feeding). Thus far, the results seem to be identical to the C57Bl6/J-BALB/c hybrids.

The process of modifying an inbred mouse strain (e.g., C57Bl/6J) onto an outbred mouse strain (e.g., CD1) is referred to as “outcrossing” or “outcross”. This is useful when mutant (knockout) animals from an inbred mouse strain show signs of weakness; offspring heterozygous for the gene of interest can be bred to an outbred mouse strain to introduce genetic variability and vigor into the inbred mouse strain. To outcross mice, the mutant mice are bred with wild type mice from the outbred strain of choice (e.g., CD1). Offspring of this breeding are hybrids (e.g., approximately: 50% C57Bl6/J and 50% CD1). Generally, no further “outcrosses” are performed as outbred mice have too much variability in their gene pool to create a congenic line.

A “domain” or “region” (used interchangeably herein) of a protein is any portion of the entire protein, up to and including the complete protein, but typically comprising less than the complete protein. A domain can, but need not, fold independently of the rest of the protein chain and/or be correlated with a particular biological, biochemical, or structural function or location (e.g., a ligand binding domain, or a cytosolic, transmembrane or extracellular domain).

“Mammal” refers to any animal classified as a mammal, including humans, domestic and farm animals, and zoo, sports, or pet animals, such as dogs, horses, cats, cows, rats, mice, non-human primates (e.g., monkeys, apes), etc.

The terms “rodent” and “rodents” refer to all members of the phylogenetic order Rodentia including any and all progeny of all future generations derived therefrom.

The term “murine” refers to any and all members of the family Muridae, including rats and mice.

The term “naturally occurring” as used throughout the specification in connection with biological materials such as polypeptides, nucleic acids, host cells, and the like, refers to materials which are found in nature.

The term “viable”, with respect to an animal, such as a mouse or particularly a global Na_{V}1.7^{-/-} knockout mouse, means that the animal is capable of reaching adulthood (in the case of a neonate or juvenile), or has reached adulthood, and is capable of living on its own with adequate nutrition.

The term “knockout” refers to partial or complete suppression of the expression of at least a portion of a protein encoded by an endogenous DNA sequence in a cell, for example, a subunit of sodium channel, voltage-gated, type IX (also known as “Na_{V}1.7”). The terms “Na_{V}1.7 knockout”, “Na_{V}1.7 KO”, “Na_{V}1.7^{-/-}”, “Na_{V}1.7^{-/-} knockout” and “Na_{V}1.7 null mutant”, are used interchangeably herein, to denote a cell or mammal exhibiting complete suppression of expression of functional Na_{V}1.7 protein. The term “hNa_{V}1.7” means human Na_{V}1.7.

The term “knockout construct” refers to a nucleic acid sequence that is designed to decrease or suppress expression of a protein encoded by endogenous DNA sequences in a cell. The nucleic acid sequence used as the knockout construct is typically comprised of (1) DNA from some portion of the gene (exon sequence, intron sequence, and/or promoter sequence) to be suppressed and (2) a marker sequence used to detect the presence of the knockout construct in the cell. The knockout construct is inserted into a cell, and integrates with the genomic DNA of the cell in such a position so as to prevent or interrupt transcription of the native DNA sequence. Such insertion usually occurs by homologous recombination (i.e., regions of the knockout construct that are homologous to endogenous DNA sequences hybridize to each other when the knockout construct is inserted into the cell and recombine so that the knockout construct is incorporated into the corresponding position of the endogenous DNA). The knockout construct nucleic acid sequence may comprise 1) a full or partial sequence of one or more exons and/or introns of the gene to be suppressed, 2) a full or partial promoter sequence of the gene to be suppressed, or 3) combinations thereof.

Typically, the knockout construct is inserted into an embryonic stem cell (ES cell) and is integrated into the ES cell genomic DNA, usually by the process of homologous recombination. This ES cell is then injected into, and integrates with, the developing embryo.

The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, many progeny of the cell will no longer express the gene at least in some cells, or will express it at a decreased level, as the DNA is now disrupted by the antibiotic resistance gene.

The term “transgene” refers to an isolated nucleotide sequence, originating in a different species from the host, that may be inserted into one or more cells of a mammal or mammalian embryo. The transgene optionally may be operably linked to other genetic elements (such as a promoter, poly A sequence and the like) that may serve to modulate, either directly, or indirectly in conjunction with the cellular machinery, the transcription and/or expression of the transgene. Alternatively or additionally, the transgene may be linked to nucleotide sequences that aid in integration of the transgene into the chromosomal DNA of the mammalian cell or embryo nucleus (as for example, in homologous recombination). The transgene may be comprised of a nucleotide sequence that is either homologous or heterologous to a particular nucleotide sequence in the mammal's endogenous genetic material, or is a hybrid sequence (i.e. one or more portions of the transgene are homologous, and one or more portions are heterologous to the mammal's genetic material). The transgene nucleotide sequence may encode a polypeptide or a variant of a polypeptide, found endogenously in the mammal, it may encode a polypeptide not naturally occurring in the mammal (i.e. an exogenous polypeptide), or it may encode a hybrid of endogenous and exogenous polypeptides. Where the transgene is operably linked to a promoter, the promoter may be homologous or heterologous to the mammal and/or to the transgene. Alternatively, the promoter may be a hybrid of endogenous and exogenous promoter elements (enhancers, silencers, suppressors, and the like).

A “pain-associated response” is any behavior recognized as typically being exhibited in a particular species of mammal when a pain-inducing stimulus is applied, e.g., paw lifting, paw licking, flinching, vocalization, or a combination of any of these, in mice and rats. In human subjects, for example, a verbal or written self-report of pain or a vocal exclamation can be a “pain associated response”.

The term “progeny” refers to any and all future generations derived and descending from a particular mammal, i.e., a mammal containing a knockout construct inserted into its genomic DNA. Thus, progeny of any successive generation are included herein such that the progeny, the F1, F2, F3, generations and so on indefinitely are included in this definition.

### ADDITIONAL EMBODIMENTS

Included within the scope of this invention is a global Nav1.7 KO mouse in which one, two, or more additional genes of interest have been “knocked out”, or “knocked in” by the insertion of a gene from a mouse (which may possess a modified nucleotide sequence) or a transgene. Such mammals can be generated by repeating the procedures set forth herein for generating each “knockout” or transgenic “knock-in” construct, or by breeding to mammals, each with a single gene knocked out, to each other, and screening for those with the double, or multiple, knockout and/or knock-in genotype. The gene to be knocked out or knocked in may be any gene provided that at least some sequence information on the DNA to be disrupted or recombinantly expressed is available to use in the preparation of both the construct and the screening probes.

Selection of Knockout Gene(s).

Usually, the DNA to be used in the knockout construct will be one or more exon and/or intron regions, and/or a promoter region, but may also be a cDNA sequence provided the cDNA is sufficiently large. Generally, the DNA will be at least about 1 kilobase (kb) in length and preferably 3-4 kb in length, thereby providing sufficient complementary sequence for hybridization when the construct is introduced into the genomic DNA of the ES cell (discussed below). Typically, a gene of interest to be knocked out will be a gene that does not result in lethality when knocked out.

The DNA sequence to be used to knock out a selected gene can be obtained using methods well known in the art such as those described by Sambrook et al. (Molecular Cloning: A Laboratory Manual, Cold Spring Harbor Laboratory Press, Cold Spring Harbor, N.Y. [1989]). Such methods include, for example, screening a genomic library with a cDNA probe encoding at least a portion of the same gene in order to obtain at least a portion of the genomic sequence. Alternatively, if a cDNA sequence is to be used in a knockout construct, the cDNA may be obtained by screening a cDNA library with oligonucleotide probes or antibodies (where the library is cloned into an expression vector). If a promoter sequence is to be used in the knockout construct, synthetic DNA probes can be designed for screening a genomic library containing the promoter sequence.

Another method for obtaining the DNA to be used in the knockout construct is to manufacture the DNA sequence synthetically, using a DNA synthesizer.

The DNA sequence encoding the knockout construct must be generated in sufficient quantity for genetic manipulation and insertion into ES cells. Amplification may be conducted by 1) placing the sequence into a suitable vector and transforming bacterial or other cells that can rapidly amplify the vector, 2) by PCR amplification, or 3) by synthesis with a DNA synthesizer.

Preparation of Knockout Constructs.

The DNA sequence to be used in producing the knockout construct is typically digested with a particular restriction enzyme selected to cut at a location(s) such that a new DNA sequence encoding a marker gene can be inserted in the proper position within this DNA sequence. The proper position for marker gene insertion is that which will serve to prevent expression of the native gene; this position will depend on various factors such as the restriction sites in the sequence to be cut, and whether an exon sequence or a promoter sequence, or both is (are) to be interrupted (i.e., the precise location of insertion necessary to inhibit promoter function or to inhibit synthesis of the native exon). Typically, the enzyme selected for cutting the DNA will generate a longer arm and a shorter arm, where the shorter arm is at least about 300 base pairs (bp). In some cases, it will be desirable to actually remove a portion or even all of one or more exons of the gene to be suppressed so as to keep the length of the knockout construct comparable to the original genomic sequence when the marker gene is inserted in the knockout construct. In these cases, the genomic DNA is cut with appropriate restriction endonucleases such that a fragment of the proper size can be removed.

The marker gene can be any nucleic acid sequence that is detectable and/or assayable, however typically it is an antibiotic resistance gene or other gene whose expression or presence in the genome can easily be detected. The marker gene is usually operably linked to its own promoter or to another strong promoter from any source that will be active or can easily be activated in the cell into which it is inserted; however, the marker gene need not have its own promoter attached as it may be transcribed using the promoter of the gene to be suppressed. In addition, the marker gene will normally have a polyA sequence attached to the 3′ end of the gene; this sequence serves to terminate transcription of the gene. Preferred marker genes are any antibiotic resistance gene such as neo (the neomycin resistance gene) and beta-gal (beta-galactosidase).

After the genomic DNA sequence has been digested with the appropriate restriction enzymes, the marker gene sequence is ligated into the genomic DNA sequence using methods well known to the skilled artisan and described in Sambrook et al., supra. The ends of the DNA fragments to be ligated must be compatible; this is achieved by either cutting all fragments with enzymes that generate compatible ends, or by blunting the ends prior to ligation. Blunting is done using methods well known in the art, such as for example by the use of Klenow fragment (DNA polymerase I) to fill in sticky ends.

The ligated knockout construct may be inserted directly into embryonic stem cells (discussed below), or it may first be placed into a suitable vector for amplification prior to insertion. Preferred vectors are those that are rapidly amplified in bacterial cells such as the pBluescript II SK vector (Stratagene, San Diego, Calif.) or pGEM7 (Promega Corp., Madison, Wis.).

Transfection of Embryonic Stem Cells

This invention contemplates production of knockout mammals from any species of rodent, including without limitation, rabbits, rats, hamsters, and mice. Preferred rodents include members of the Muridae family, including rats and mice. Mouse strains from which ES cells can be derived for KO generation include C57BL/6, 129SV, CD1, or BALB/c. Generally, the embryonic stem cells (ES cells) used to produce the knockout mammal will be of the same species as the knockout mammal to be generated. Thus for example, mouse embryonic stem cells will usually be used for generation of knockout mice.

Embryonic stem cells are typically selected for their ability to integrate into and become part of the germ line of a developing embryo so as to create germ line transmission of the knockout construct. Thus, any ES cell line that is believed to have this capability is suitable for use herein. One mouse strain that is typically used for production of ES cells, is the 129J strain. A preferred ES cell line is murine cell line D3 (American Type Culture Collection catalog no. CRL 1934). The cells are cultured and prepared for DNA insertion using methods well known to the skilled artisan such as those set forth by Robertson (in: Teratocarcinomas and Embryonic Stem Cells: A Practical Approach, E. J. Robertson, ed. IRL Press, Washington, D.C. [1987]) and by Bradley et al. (Current Topics in Devel. Biol., 20:357-371 [1986]) and by Hogan et al. (Manipulating the Mouse Embryo: A Laboratory Manual, Cold Spring Harbor Laboratory Press, Cold Spring Harbor, N.Y. [1986]).

Insertion of the knockout construct into the ES cells can be accomplished using a variety of methods well known in the art including for example, electroporation, microinjection, and calcium phosphate treatment (see Lovell-Badge, in Robertson, ed., supra). A preferred method of insertion is electroporation.

Each knockout construct DNA to be inserted into the cell must first be linearized if the knockout construct has been inserted into a vector. Linearization is accomplished by digesting the DNA with a suitable restriction endonuclease selected to cut only within the vector sequence and not within the knockout construct sequence.

For insertion of the DNA sequence, the knockout construct DNA is added to the ES cells under appropriate conditions for the insertion method chosen. Where more than one construct is to be introduced into the ES cell, DNA encoding each construct can be introduced simultaneously or one at a time.

If the cells are to be electroporated, the ES cells and knockout construct DNA are exposed to an electric pulse using an electroporation machine and following the manufacturer's guidelines for use. After electroporation, the cells are allowed to recover under suitable incubation conditions. The cells are then screened for the presence of the knockout construct.

Screening can be done using a variety of methods. Where the marker gene is an antibiotic resistance gene, the cells are cultured in the presence of an otherwise lethal concentration of antibiotic. Those cells that survive have presumably integrated the knockout construct. If the marker gene is other than an antibiotic resistance gene, a Southern blot of the ES cell genomic DNA can be probed with a sequence of DNA designed to hybridize only to the marker sequence. Finally, if the marker gene is a gene that encodes an enzyme whose activity can be detected (e.g., beta-galactosidase), the enzyme substrate can be added to the cells under suitable conditions, and the enzymatic activity can be analyzed.

The knockout construct may be integrated into several locations in the ES cell genome, and may integrate into a different location in each cell's genome, due to the occurrence of random insertion events; the desired location of the insertion is in a complementary position to the DNA sequence to be knocked out. Typically, less than about 1-5 percent of the ES cells that take up the knockout construct will actually integrate the knockout construct in the de sired location. To identify those cells with proper integration of the knockout construct, the DNA can be extracted from the cells using standard methods such as those described by Sambrook et al., supra. The DNA can then be probed on a Southern blot with a probe or probes designed to hybridize in a specific pattern to genomic DNA digested with (a) particular restriction enzyme(s). Alternatively, or additionally, the genomic DNA can be amplified by PCR with probes specifically designed to amplify DNA fragments of a particular size and sequence (i.e., only those cells containing the knockout construct in the proper position will generate DNA fragments of the proper size).

Injection/Implantation of Embryos.

After suitable ES cells containing the knockout construct in the proper location have been identified, the cells are inserted into an embryo. Insertion may be accomplished in a variety of ways, however a preferred method is by microinjection. For microinjection, about 10-30 cells are collected into a micropipet and injected into embryos that are at the proper stage of development to integrate the ES cell into the developing embryo.

The suitable stage of development for the embryo is very species dependent, however for mice it is about 3.5 days. The embryos are obtained by perfusing the uterus of pregnant females. Suitable methods for accomplishing this are known to the skilled artisan, and are set forth by Bradley (in Robertson, ed., supra).

While any embryo of the right age/stage of development is suitable for use, preferred embryos are male and have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo is implanted into the uterus of a pseudopregnant foster mother. While any foster mother may be used, they are typically selected for their ability to breed and reproduce well, and for their ability to care for their young. Such foster mothers are typically prepared by mating with vasectomized males of the same species. The stage of the pseudopregnant foster mother is important for successful implantation, and it is species dependent. For mice, this stage is about 2-3 days pseudopregnant.

Screening for Presence of Knockout Gene.

In general, offspring that are born to the foster mother may be screened initially for mosaic coat color where the coat color selection strategy (as described above) has been employed. In addition, or as an alternative, DNA from tail tissue of the offspring may be screened for the presence of the knockout construct using Southern blots and/or PCR as described above. Offspring that appear to be mosaics are then crossed to each other if they are believed to carry the knockout construct in their germ line to generate homozygous knockout animals. If it is unclear whether the offspring will have germ line transmission, they can be crossed with a parental or other strain and the offspring screened for heterozygosity. The heterozygotes are identified by Southern blots and/or PCR amplification of the DNA, as set forth above.

The heterozygotes can then be crossed with each other to generate homozygous knockout offspring. Homozygotes may be identified by Southern blotting of equivalent amounts of genomic DNA from mice that are the product of this cross, as well as mice that are known heterozygotes and wild type mice. Probes to screen the Southern blots can be designed as set forth above.

Other means of identifying and characterizing the knockout offspring are available. For example, Northern blots can be used to probe the mRNA for the presence or absence of transcripts encoding either the gene knocked out, the marker gene, or both. In addition, Western blots can be used to assess the level of expression of the gene knocked out in various tissues of these offspring by probing the Western blot with an antibody against the protein encoded by the gene knocked out, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Because neonatal Na_{V}1.7 KO mice were reported to exhibit an apparent failure to feed (Nassar et al., 2004), hand feeding can be used to provide nutrition to neonatal Na_{V}1.7 KO mice, as described in Example 1 herein. An alternative feeding method/artificial rearing, that does not require “hand feeding” can also be useful, such as using intravenous feeding or gastric implants and syringe pumps; however, the necessary surgery involved poses considerable risks to new born mice. Alternatively, the so-called “pup in a cup” technique can be used (West, Use of Pup in a Cup Model to Study Brain Development, J. Nutr., 123:382-385 (1993)), involving raising each mouse pup singly in a cup. However, in addition to physical injury that may be caused by gastric surgery related to the syringe pump involved, this technique might induce behavioral problems in these social animals, therefore affecting the reliability of some in vivo data obtained using such mice. Another possible way to increase survival of knockouts, avoiding such aforementioned complications, is to place knockouts with a lactating dam, in which lactation has been induced by a normal litter of mouse pups. That is, Na_{V}1.7 knockout mouse pups are swapped for the normal litter as needed for feeding the Na_{V}1.7 knockout mouse pups. To ensure the dam does not reject the foreign Na_{V}1.7 KO mouse pups, knockouts can be marked with the scent of the dam. Occasional bloating has been observed in neonates of all genotypes. This bloating is characterized by the presence of air in the gastric cavity leading to distension of the abdomen. In such circumstances, air can be removed manually using an ultra fine insulin syringe fitted with a permanently attached 29 gauge ½ inch needle.

Transgene Technology

Selection of Transgene(s).

Typically, the transgene(s) useful in the present invention will be a nucleotide sequence encoding a polypeptide of interest, e.g., a polypeptide involved in the nervous system, an immune response, hematopoiesis, inflammation, cell growth and proliferation, cell lineage differentiation, and/or the stress response. Included within the scope of this invention is the insertion of one, two, or more transgenes into a Na_{V}1.7 knockout mouse of the invention.

Where more than one transgene is used in this invention, the transgenes may be prepared and inserted individually, or may be generated together as one construct for insertion. The transgenes may be homologous or heterologous to both the promoter selected to drive expression of each transgene and/or to the mammal. Further, the transgene may be a full length cDNA or genomic DNA sequence, or any fragment, subunit or mutant thereof that has at least some biological activity i.e., exhibits an effect at any level (biochemical, cellular and/or morphological) that is not readily observed in a wild type, non-transgenic mammal of the same species. Optionally, the transgene may be a hybrid nucleotide sequence, i.e., one constructed from homologous and/or heterologous cDNA and/or genomic DNA fragments. The transgene may also optionally be a mutant of one or more naturally occurring cDNA and/or genomic sequences, or an allelic variant thereof.

Each transgene may be isolated and obtained in suitable quantity using one or more methods that are well known in the art. These methods and others useful for isolating a transgene are set forth, for example, in Sambrook et al. (Molecular Cloning: A Laboratory Manual, Cold Spring Harbor Laboratory Press, Cold Spring Harbor, N.Y. [1989]) and in Berger and Kimmel (Methods in Enzymology: Guide to Molecular Cloning Techniques, vol. 152, Academic Press, Inc., San Diego, Calif. [1987]).

Where the nucleotide sequence of each transgene is known, the transgene may be synthesized, in whole or in part, using chemical synthesis methods such as those described in Engels et al. (Angew. Chem. Int. Ed. Engl., 28:716-734 [1989]). These methods include, inter alia, the phosphotriester, phosphoramidite and H-phosphonate methods of nucleic acid synthesis. Alternatively, the transgene may be obtained by screening an appropriate cDNA or genomic library using one or more nucleic acid probes (oligonucleotides, cDNA or genomic DNA fragments with an acceptable level of homology to the transgene to be cloned, and the like) that will hybridize selectively with the transgene DNA. Another suitable method for obtaining a transgene is the polymerase chain reaction (PCR). However, successful use of this method requires that enough information about the nucleotide sequence of the transgene be available so as to design suitable oligonucleotide primers useful for amplification of the appropriate nucleotide sequence.

Where the method of choice requires the use of oligonucleotide primers or probes (e.g. PCR, cDNA or genomic library screening), the oligonucleotide sequences selected as probes or primers should be of adequate length and sufficiently unambiguous so as to minimize the amount of non-specific binding that will occur during library screening or PCR. The actual sequence of the probes or primers is usually based on conserved or highly homologous sequences or regions from the same or a similar gene from another organism. Optionally, the probes or primers can be degenerate.

In cases where only the amino acid sequence of the transgene is known, a probable and functional nucleic acid sequence may be inferred for the transgene using known and preferred codons for each amino acid residue. This sequence can then be chemically synthesized.

This invention encompasses the use of transgene mutant sequences. A mutant transgene is a transgene containing one or more nucleotide substitutions, deletions, and/or insertions as compared to the wild type sequence. The nucleotide substitution, deletion, and/or insertion can give rise to a gene product (i.e., protein) that is different in its amino acid sequence from the wild type amino acid sequence. Preparation of such mutants is well known in the art, and is described for example in Wells et al. (Gene, 34:315 [1985]), and in Sambrook et al, supra.

Selection of Regulatory Elements.

Transgenes are typically operably linked to promoters, where a promoter is selected to regulate expression of each transgene in a particular manner.

Where more than one transgene is to be used, each transgene may be regulated by the same or by a different promoter. The selected promoters may be homologous (i.e., from the same species as the mammal to be transfected with the transgene) or heterologous (i.e., from a source other than the species of the mammal to be transfected with the transgene). As such, the source of each promoter may be from any unicellular, prokaryotic or eukaryotic organism, or any vertebrate or invertebrate organism.

Selection of Other Vector Components

In addition to the transgene and the promoter, the vectors useful for preparing the transgenes of this invention typically contain one or more other elements useful for (1) optimal expression of transgene in the mammal into which the transgene is inserted, and (2) amplification of the vector in bacterial or mammalian host cells. Each of these elements will be positioned appropriately in the vector with respect to each other element so as to maximize their respective activities. Such positioning is well known to the ordinary skilled artisan. The following elements may be optionally included in the vector as appropriate.

i. Signal Sequence Element

For those embodiments of the invention where the polypeptide encoded by the transgene is to be secreted, a small polypeptide termed signal sequence is frequently present to direct the polypeptide encoded by the transgene out of the cell where it is synthesized. Typically, the signal sequence is positioned in the coding region of the transgene towards or at the 5′ end of the coding region. Many signal sequences have been identified, and any of them that are functional and thus compatible with the transgenic tissue may be used in conjunction with the transgene. Therefore, the nucleotide sequence encoding the signal sequence may be homologous or heterologous to the transgene, and may be homologous or heterologous to the transgenic mammal. Additionally, the nucleotide sequence encoding the signal sequence may be chemically synthesized using methods set forth above. However, for purposes herein, preferred signal sequences are those that occur naturally with the transgene (i.e., are homologous to the transgene).

ii. Membrane Anchoring Domain Element

In some cases, it may be desirable to have a transgene expressed on the surface of a particular intracellular membrane or on the plasma membrane. Naturally occurring membrane proteins contain, as part of the polypeptide, a stretch of amino acids that serve to anchor the protein to the membrane. However, for proteins that are not naturally found on the membrane, such a stretch of amino acids may be added to confer this feature. Frequently, the anchor domain will be an internal portion of the polypeptide sequence and thus the nucleotide sequence encoding it will be engineered into an internal region of the transgene nucleotide sequence. However, in other cases, the nucleotide sequence encoding the anchor domain may be attached to the 5′ or 3′ end of the transgene nucleotide sequence. Here, the nucleotide sequence encoding the anchor domain may first be placed into the vector in the appropriate position as a separate component from the nucleotide sequence encoding the transgene. As for the signal sequence, the anchor domain may be from any source and thus may be homologous or heterologous with respect to both the transgene and the transgenic mammal. Alternatively, the anchor domain may be chemically synthesized using methods set forth above.

iii. Origin of Replication Element

This component is typically a part of prokaryotic expression vectors purchased commercially, and aids in the amplification of the vector in a host cell. If the vector of choice does not contain an origin of replication site, one may be chemically synthesized based on a known sequence, and ligated into the vector.

iv. Transcription Termination Element

This element, also known as the polyadenylation or polyA sequence, is typically located 3′ to the transgene nucleotide sequence in the vector, and serves to terminate transcription of the transgene. While the nucleotide sequence encoding this element is easily cloned from a library or even purchased commercially as part of a vector, it can also be readily synthesized using methods for nucleotide sequence synthesis such as those described above.

v. Intron Element

In many cases, transcription of the transgene is increased by the presence of one intron or more than one intron (linked by exons) on the cloning vector. The intron(s) may be naturally occurring within the transgene nucleotide sequence, especially where the transgene is a full length or a fragment of a genomic DNA sequence. Where the intron(s) is not naturally occurring within the nucleotide sequence (as for most cDNAs), the intron(s) may be obtained from another source. The intron(s) may be homologous or heterologous to the transgene and/or to the transgenic mammal. The position of the intron with respect to the promoter and the transgene is important, as the intron must be transcribed to be effective. As such, where the transgene is a cDNA sequence, the preferred position for the intron(s) is 3′ to the transcription start site, and 5′ to the polyA transcription termination sequence. Preferably for cDNA transgenes, the intron will be located on one side or the other (i.e., 5′ or 3′) of the transgene nucleotide sequence such that it does not interrupt the transgene nucleotide sequence. Any intron from any source, including any viral, prokaryotic and eukaryotic (plant or animal) organisms, may be used to practice this invention, provided that it is compatible with the host cell(s) into which it is inserted. Also included herein are synthetic introns. Optionally, more than one intron may be used in the vector. A preferred set of introns and exons is the human growth hormone (hGH) DNA sequence.

vi. Selectable Marker(s) Element

Selectable marker genes encode polypeptides necessary for the survival and growth of transfected cells grown in a selective culture medium. Typical selection marker genes encode proteins that (a) confer resistance to antibiotics or other toxins, e.g., ampicillin, tetracycline, or kanomycin for prokaryotic host cells, and neomycin, hygromycin, or methotrexate for mammalian cells; (b) complement auxotrophic deficiencies of the cell; or (c) supply critical nutrients not available from complex media, e.g., the gene encoding D-alanine racemase for cultures of Bacilli.

All of the elements set forth above, as well as others useful in this invention, are well known to the skilled artisan and are described, for example, in Sambrook et al. (Molecular Cloning: A Laboratory Manual, Cold Spring Harbor Laboratory Press, Cold Spring Harbor, N.Y. [1989]) and Berger et al., eds. (Guide to Molecular Cloning Techniques, Academic Press, Inc., San Diego, Calif. [1987]).

Construction of Cloning Vectors

The cloning vectors most useful for amplification of transgene cassettes useful in preparing the transgenic mammals of this invention are those that are compatible with prokaryotic cell hosts. However, eukaryotic cell hosts, and vectors compatible with these cells, are within the scope of the invention.

In certain cases, some of the various elements to be contained on the cloning vector may be already present in commercially available cloning or amplification vectors such as pUC18, pUC19, pBR322, the pGEM vectors (Promega Corp, Madison, Wis.), the pBluescript® vectors such as pBIISK+/− (Stratagene Corp., La Jolla, Calif.), and the like, all of which are suitable for prokaryotic cell hosts. In this case it is necessary to only insert the transgene(s) into the vector.

However, where one or more of the elements to be used are not already present on the cloning or amplification vector, they may be individually obtained and ligated into the vector. Methods used for obtaining each of the elements and ligating them are well known to the skilled artisan and are comparable to the methods set forth above for obtaining a transgene (i.e., synthesis of the DNA, library screening, and the like).

Vectors used for cloning or amplification of the transgene(s) nucleotide sequences and/or for transfection of the mammalian embryos are constructed using methods well known in the art. Such methods include, for example, the standard techniques of restriction endonuclease digestion, ligation, agarose and acrylamide gel purification of DNA and/or RNA, column chromatography purification of DNA and/or RNA, phenol/chloroform extraction of DNA, DNA sequencing, polymerase chain reaction amplification, and the like, as set forth in Sambrook et al., supra.

The final vector used to practice this invention is typically constructed from a starting cloning or amplification vector such as a commercially available vector. This vector may or may not contain some of the elements to be included in the completed vector. If none of the desired elements are present in the starting vector, each element may be individually ligated into the vector by cutting the vector with the appropriate restriction endonuclease(s) such that the ends of the element to be ligated in and the ends of the vector are compatible for ligation. In some cases, it may be necessary to “blunt” the ends to be ligated together in order to obtain a satisfactory ligation. Blunting is accomplished by first filling in “sticky ends” using Klenow DNA polymerase or T4 DNA polymerase in the presence of all four nucleotides. This procedure is well known in the art and is described for example in Sambrook et al., supra.

Alternatively, two or more of the elements to be, inserted into the vector may first be ligated together (if they are to be positioned adjacent to each other) and then ligated into the vector.

One other method for constructing the vector is to conduct all ligations of the various elements simultaneously in one reaction mixture. Here, many nonsense or nonfunctional vectors will be generated due to improper ligation or insertion of the elements, however the functional vector may be identified and selected by restriction endonuclease digestion.

After the vector has been constructed, it may be transfected into a prokaryotic host cell for amplification. Cells typically used for amplification are E coli DH5-alpha (Gibco/BRL, Grand. Island, N.Y.) and other E. coli strains with characteristics similar to DH5-alpha.

Where mammalian host cells are used, cell lines such as Chinese hamster ovary (CHO cells; Urlab et al., Proc. Natl. Acad. Sci. USA, 77:4216 [1980])) and human embryonic kidney cell line 293 (Graham et al., J. Gen. Virol., 36:59 [1977]), as well as other lines, are suitable.

Transfection of the vector into the selected host cell line for amplification is accomplished using such methods as calcium phosphate, electroporation, microinjection, lipofection or DEAE-dextran. The method selected will in part be a function of the type of host cell to be transfected. These methods and other suitable methods are well known to the skilled artisan, and are set forth in Sambrook et al., supra.

After culturing the cells long enough for the vector to be sufficiently amplified (usually overnight for E. coli cells), the vector (often termed plasmid at this stage) is isolated from the cells and purified. Typically, the cells are lysed and the plasmid is extracted from other cell contents. Methods suitable for plasmid purification include inter alia, the alkaline lysis mini-prep method (Sambrook et al., supra).

Preparation of Plasmid for Insertion

Typically, the plasmid containing the transgene is linearized, and portions of it removed using a selected restriction endonuclease prior to insertion into the embryo. In some cases, it may be preferable to isolate the transgene, promoter, and regulatory elements as a linear fragment from the other portions of the vector, thereby injecting only a linear nucleotide sequence containing the transgene, promoter, intron (if one is to be used), enhancer, polyA sequence, and optionally a signal sequence or membrane anchoring domain into the embryo. This may be accomplished by cutting the plasmid so as to remove the nucleic acid sequence region containing these elements, and purifying this region using agarose gel electrophoresis or other suitable purification methods.

Production of Transgenic or Knockout Mammals

Transgenic or knockout (KO) mammals may readily be prepared using methods well known to the skilled artisan. For example, to prepare transgenic rodents, methods such as those set forth by Hogan et al., eds., (Manipulating the Mouse Embryo: A Laboratory Manual, Cold Spring Harbor Laboratory Press, Cold Spring Harbor, N.Y. [1986]) may be employed.

The specific line(s) of any mammalian species used to practice this invention are selected for general good health, good embryo yields, good pronuclear visibility in the embryos, and good reproductive fitness. In addition, the haplotype is a significant factor. For example, when transgenic mice are to be produced, strains such as C57BL/6 or C57BL/6.times.DBA/2 F.sub.1, or FVB lines are often used (obtained commercially from Charles River Labs, Boston, Mass., The Jackson Laboratory, Bar Harbor, Me., or Taconic Labs.). Preferred strains are those with H-2.sup.b, H-2.sup.d or H-2.sup.q haplotypes such as C57BL/6 or DBA/1. The line(s) used to practice this invention may themselves be transgenics, and/or may be knockouts (i.e., mammals which have one or more genes partially or completely suppressed). Preferably the same line will be used for preparation of both the initial knockout mammals and the transgenic mammals. This will make subsequent breeding and backcrossing more efficient.

The creation of a knockout (KO) mouse line (for a desired gene) starts with the implantation of modified embryonic stem (ES) cells from one mouse strain “X” into a blastocyst of a different mouse strain “Y”. It is generally advantageous to choose mouse strains of different coat color to obtain an animal bearing a “chimeric” coat composed of both colors. For example, if the modified ES cells come from an agouti mouse line (129/Sv) and are inserted into a black mouse blastocyst (C57/Bl6), one will generate chimeras with agouti and black coat. The next crucial step is to determine whether the modified ES cells have populated the gonads for germ line transmission. This is determined by crossing the chimeric mice to animals of the strain corresponding to the blastocyst source (C57/Bl6). If the cross generates pups that are agouti (ES cell color), it shows that germline transmission was achieved in a C57Bl6/129Sv mixed background.

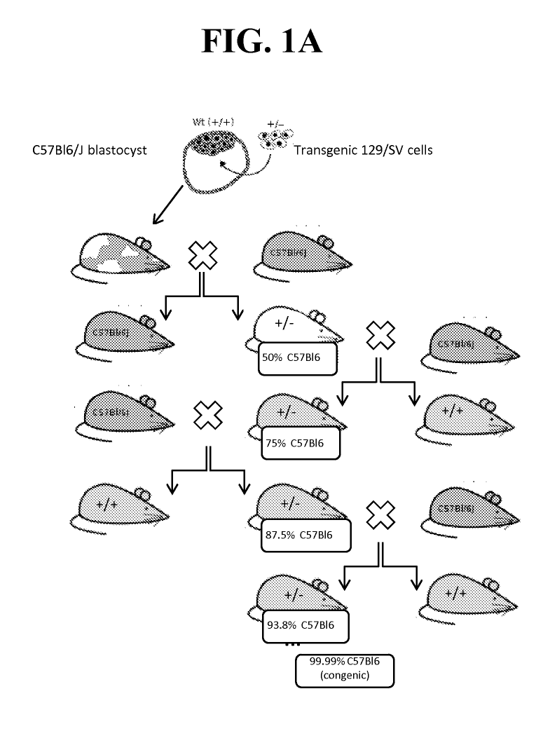

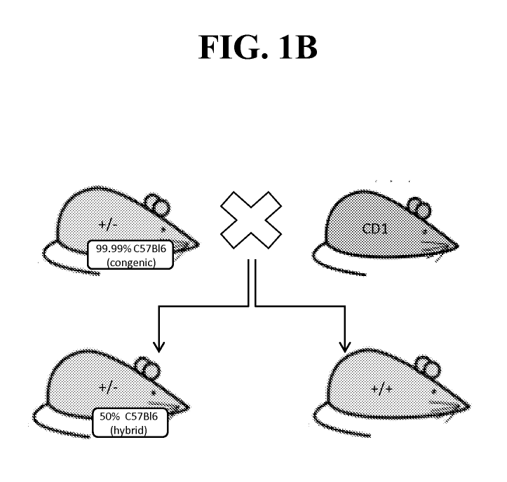

A mixed background is not ideal in genetic studies, as different mouse strains behave differently. It is therefore preferable to create a pure mouse line from one specific mouse strain. When breeding to the mouse of interest, backcrossing is used with an inbred mouse line and outcrossing is used with an outbred mouse line. Backcrossing is performed as depicted schematically in FIG. 1A until one reaches a ˜99.99% genetic identity with the selected inbred mouse line; this takes approximately 8 to 10 backcrosses and is the preferred approach for research purposes. This new line is now considered congenic. Outcrossing (shown schenmatically in FIG. 1B) only involves one cross and one obtains a hybrid mouse carrying half the genetic code of each parental line. This line is not considered congenic and cannot be made into a congenic line due to the substantial genetic variability of the lines. Unfortunately, as is typical with inbred lines, backcrossing may make the resultant mouse colony weaker and prone to recessive genetic disease. Outcrossing can't generate a pure line as it is performed only once (1× cross) and is meant to insert genetic diversity in a highly inbred mouse line. It often restores fertility, vigor and size to breeding lines.

The age of the mammals that are used to obtain embryos and to serve as surrogate hosts is a function of the species used, but is readily determined by one of ordinary skill in the art. For example, when mice are used, pre-puberal females are preferred, as they yield more embryos and respond better to hormone injections.

Similarly, the male mammal to be used as a stud will normally be selected by age of sexual maturity, among other criteria.

Administration of hormones or other chemical compounds may be necessary to prepare the female for egg production, mating, and/or reimplantation of embryos. The type of hormones/cofactors and the quantity used, as well as the timing of administration of the hormones will vary for each species of mammal. Such considerations will be readily apparent to one of ordinary skill in the art.

Typically, a primed female (i.e., one that is producing eggs that can be fertilized) is mated with a stud male, and the resulting fertilized embryos are then removed for introduction of the transgene(s). Alternatively, eggs and sperm may be obtained from suitable females and males and used for in vitro fertilization to produce an embryo suitable for introduction of the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method is to incubate the embryos in vitro for about 1-7 days, depending on the species, and then reimplant them into the surrogate host.

Reimplantation is accomplished using standard methods. The female “foster mother” strain to be used is selected for general hardiness and health, and for her ability to care for the offspring. In the case of mice, strains such as C57BL/6.times.DBA1 or CD1, or BALB/c are generally suitable. However, for the Nav1.7−/− knockouts of the present invention, a C57BL/6 background is particularly not well suited, because the pups lack sufficient vigor and the dams are typically not sufficiently diligent mothers.

Usually, the surrogate host is anesthetized, and the embryos are inserted into the oviduct. The number of embryos implanted into a particular host will vary by species, but will usually be comparable to the number of offspring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue (about 1 cm is removed from the tip of the tail) and analyzed by Southern analysis or PCR for the transgene. Alternatively, the tissues or cells believed to express the transgene at the highest levels are tested for the presence and expression of the transgene using Southern analysis or PCR, although any tissues or cell types may e used for this analysis.

Alternative or additional methods for evaluating the presence of the transgene include, without limitation, suitable biochemical assays such as enzyme and/or immunological assays, histological stains for particular markers or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic mammals may be obtained by mating the transgenic mammal with a suitable partner, or by in vitro fertilization of eggs and/or sperm obtained from the transgenic mammal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

Preparation of Knockout/Transgenic Mammals

Mammals containing more than one knockout construct and/or more than one transgene are prepared in any of several ways. Typically, the manner of preparation is to generate a series of mammals, e.g., a mouse, each containing one of the desired knockout constructs or transgenes, as described herein. Such mammals are bred together through a series of crosses, backcrosses and selections, to ultimately generate a single mammal containing all desired knockout constructs and/or transgenes, where the mammal is otherwise congenic (genetically identical) to the wild type except for the presence of the knockout(s) constructs and/or transgene(s).

Typically, crossing and backcrossing is accomplished by mating siblings or a parental strain with an offspring, depending on the goal of each particular step in the breeding process. In certain cases, it may be necessary to generate a large number of offspring in order to generate a single offspring that contains each of the knockout constructs and/or transgenes in the proper chromosomal location. In addition, it may be necessary to cross or backcross over several generations to ultimately obtain the desired genotype.

Uses of Knockout Mammals

In general, knockout mammals have a variety of uses depending on the gene or genes that have been suppressed. For example, where the gene or genes suppressed encode proteins believed to be involved in immunosuppression or inflammation, the mammal may be used to screen for drugs useful for immunomodulation, i.e., drugs that either enhance or inhibit these activities.

The global Na_{V}1.7 knockout mice of the invention can be used to screen potential drugs for the treatments of pain, neuroendocrine disorders, or prostate cancer. Screening for useful drugs would involve administering the candidate drug over a range of doses to the mouse, and assaying at various time points for the effect(s) of the drug on the disorder being evaluated. In addition, mammals of the present invention can be useful for evaluating the development of the nervous system, and for studying the effects of particular Na_{V}1.7 gene mutations. Embodiments of the Na_{V}1.7 knockout mice and its progeny of this invention will also have a variety of uses depending on the additional transgenes that can be expressed and/or the knockout constructs they may contain. Screening for a useful drug would involve first inducing the disease, or inducing a model of the disease, in the mammal and then administering the candidate drug over a range of doses to the mammal, and assaying at various time points for the effect(s) of the drug on the disease or disorder being evaluated. Alternatively, or additionally, the drug could be administered prior to or simultaneously with exposure to induction of the disease or disease model. In other embodiments, the inventive global Na_{V}1.7^{-/-} knockout mice are further useful for drug research and development, for example, in in vivo protocols to distinguish on-target/off-target effects or distinguish between pain and sedation effects.

In addition to screening a drug for use in treating a disease or condition, the mammal of the present invention could be useful in designing a therapeutic regimen aimed at preventing or curing the disease or condition. For example, the mammal might be treated with a combination of a particular diet, exercise routine, radiation treatment, and/or one or more compounds or substances either prior to, or simultaneously, or after, the onset of the disease or condition. Such an overall therapy or regimen might be more effective at combating the disease or condition than treatment with a compound alone. In addition, such criteria as blood pressure, body temperature, body weight, pulse, behavior, appearance of coat (ruffled fur) and the like could be evaluated.

The global Na_{V}1.7^{-/-} knockout mice of this invention may also be used to generate one or more cell lines. Such cell lines have many uses, as for example, to evaluate the effect(s) of the knockout on a particular tissue or organ, and to screen compounds that may affect the level of activity of the Na_{V}1.7 in the tissue. Such compounds may be useful as therapeutics.

Production of such cell lines may be accomplished using a variety of methods, known to the skilled artisan. The actual culturing conditions will depend on the tissue and type of cells to be cultured. Various media containing different concentrations of macro and micro nutrients, growth factors, serum, and the like, can be tested on the cells without undue experimentation to determine the optimal conditions for growth and proliferation of the cells. Similarly, other culturing conditions such as cell density, media temperature, and carbon dioxide concentrations in the incubator can also readily be evaluated and optimized, and identifying compounds that affect this process.

Other uses will be readily apparent to one of skill in the art, including the preparation of antibodies against Na_{V}1.7, including murine or human Na_{V}1.7, because Na_{V}1.7 KO mice are not self-tolerant against murine Nav1.7 or sequences of other species of Nav1.7 that are closely related, such as the human SCN9A gene product. Based on the CDR sequences of the anti-human Na_{V}1.7 antibodies produced by the inventive knockout mice, chimeric or humanized antibodies can be developed incorporating those CDRs into an antibody for either antagonizing or agonizing Na_{V}1.7 ion channel activity. The therapeutic value of an antagonistic or blocking anti-human Na_{V}1.7 antibody is readily apparent to one of skill in the art.

Production of Antibodies

Polyclonal Antibodies.

Polyclonal antibodies are typically raised in animals by multiple subcutaneous (sc) or intraperitoneal (ip) injections of the relevant antigen and an adjuvant. Alternatively, antigen may be injected directly into the animal's lymph node (see Kilpatrick et al., Hybridoma, 16:381-389, 1997). An improved antibody response may be obtained by conjugating the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride or other agents known in the art.

Animals are immunized against the antigen, immunogenic conjugates, or derivatives by combining, e.g., 100 μg of the protein or conjugate (for mice) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. At 7-14 days post-booster injection, the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal Antibodies.

Monoclonal antibodies can be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. For example, monoclonal antibodies can be made using the hybridoma method first described by Kohler et al., Nature, 256:495 (1975), or can be made by recombinant DNA methods (e.g., Cabilly et al., Methods of producing immunoglobulins, vectors and transformed host cells for use therein, U.S. Pat. No. 6,331,415), including methods, such as the “split DHFR” method, that facilitate the generally equimolar production of light and heavy chains, optionally using mammalian cell lines (e.g., CHO cells) that can glycosylate the antibody (See, e.g., Page, Antibody production, EP0481790 A2 and U.S. Pat. No. 5,545,403).

In the hybridoma method, a mouse or other appropriate host mammal, such as rats, hamster or macaque monkey, is immunized as herein described to elicit lymphocytes that produce or are capable of producing antibodies that will specifically bind to the protein used for immunization. Alternatively, lymphocytes can be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells, once prepared, are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)). Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by BIAcore® or Scatchard analysis (Munson et al., Anal. Biochem., 107:220 (1980); Fischer et al., A peptide-immunoglobulin-conjugate, WO 2007/045463 A1, Example 10, which is incorporated herein by reference in its entirety).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

Hybridomas or mAbs may be further screened to identify mAbs with particular properties, such as binding affinity with a particular antigen or target. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, affinity chromatography, or any other suitable purification technique known in the art.

Recombinant Production of Antibodies and Other Polypeptides.

Relevant amino acid sequences from an immunoglobulin or polypeptide of interest may be determined by direct protein sequencing, and suitable encoding nucleotide sequences can be designed according to a universal codon table. Alternatively, genomic or cDNA encoding the monoclonal antibodies may be isolated and sequenced from cells producing such antibodies using conventional procedures (e.g., by using oligonucleotide probes that are capable of binding specifically to genes encoding the heavy and light chains of the monoclonal antibodies). Relevant DNA sequences can be determined by direct nucleic acid sequencing.

Cloning of DNA is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library may be constructed by reverse transcription of polyA+ mRNA, preferably membrane-associated mRNA, and the library screened using probes specific for human immunoglobulin polypeptide gene sequences. In one embodiment, however, the polymerase chain reaction (PCR) is used to amplify cDNAs (or portions of full-length cDNAs) encoding an immunoglobulin gene segment of interest (e.g., a light or heavy chain variable segment). The amplified sequences can be readily cloned into any suitable vector, e.g., expression vectors, minigene vectors, or phage display vectors. It will be appreciated that the particular method of cloning used is not critical, so long as it is possible to determine the sequence of some portion of the immunoglobulin polypeptide of interest.

One source for antibody nucleic acids is a hybridoma produced by obtaining a B cell from an animal immunized with the antigen of interest and fusing it to an immortal cell. Alternatively, nucleic acid can be isolated from B cells (or whole spleen) of the immunized animal. Yet another source of nucleic acids encoding antibodies is a library of such nucleic acids generated, for example, through phage display technology. Polynucleotides encoding peptides of interest, e.g., variable region peptides with desired binding characteristics, can be identified by standard techniques such as panning.

The sequence encoding an entire variable region of the immunoglobulin polypeptide may be determined; however, it will sometimes be adequate to sequence only a portion of a variable region, for example, the CDR-encoding portion. Sequencing is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74: 5463-5467, which is incorporated herein by reference). By comparing the sequence of the cloned nucleic acid with published sequences of human immunoglobulin genes and cDNAs, one of skill will readily be able to determine, depending on the region sequenced, (i) the germline segment usage of the hybridoma immunoglobulin polypeptide (including the isotype of the heavy chain) and (ii) the sequence of the heavy and light chain variable regions, including sequences resulting from N-region addition and the process of somatic mutation. One source of immunoglobulin gene sequence information is the National Center for Biotechnology Information, National Library of Medicine, National Institutes of Health, Bethesda, Md.

Isolated DNA can be operably linked to control sequences or placed into expression vectors, which are then transfected into host cells that do not otherwise produce immunoglobulin protein, to direct the synthesis of monoclonal antibodies in the recombinant host cells. Recombinant production of antibodies is well known in the art.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Many vectors are known in the art. Vector components may include one or more of the following: a signal sequence (that may, for example, direct secretion of the antibody; e.g., ATGGACATGAGGGTGCCCGCTCAGCTCCTGGGGCTCCTGCTGCTGTGGCT GAGAGGTGCGCGCTGT// SEQ ID NO:4, which encodes the VK-1 signal peptide sequence MDMRVPAQLLGLLLLWLRGARC// SEQ ID NO:5), an origin of replication, one or more selective marker genes (that may, for example, confer antibiotic or other drug resistance, complement auxotrophic deficiencies, or supply critical nutrients not available in the media), an enhancer element, a promoter, and a transcription termination sequence, all of which are well known in the art.

Cell, cell line, and cell culture are often used interchangeably and all such designations herein include progeny. Transformants and transformed cells include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus Salmonella, e.g., Salmonella typhimurium, Serratia, e.g., Serratia marcescans, and Shigella, as well as Bacillus such as B. subtilis and B. licheniformis, Pseudomonas, and Streptomyces. Eukaryotic microbes such as filamentous fungi or yeast are suitable cloning or expression hosts for recombinant polypeptides or antibodies. Saccharomyces cerevisiae, or common baker's yeast, is the most commonly used'among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Host cells for the expression of glycosylated antibodies can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of polypeptides (including antibody) from such cells has become routine procedure. Examples of useful mammalian host cell lines are Chinese hamster ovary cells, including CHOK1 cells (ATCC CCL61), DXB-11, DG-44, and Chinese hamster ovary cells/−DHFR(CHO, Urlaub et al., Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, [Graham et al., J. Gen Virol. 36: 59 (1977)]; baby hamster kidney cells (BHK, ATCC CCL 10); mouse sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TR1 cells (Mather et al., Annals N.Y. Acad. Sci. 383: 44-68 (1982)); MRC 5 cells or FS4 cells; or mammalian myeloma

Host cells are transformed or transfected with the above-described nucleic acids or vectors for production of polypeptides (including antibodies) and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of polypeptides, such as antibodies.

The host cells used to produce the polypeptides useful in the invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. No. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Upon culturing the host cells, the recombinant polypeptide can be produced intracellularly, in the periplasmic space, or directly secreted into the medium. If the polypeptide, such as an antibody, is produced intracellularly, as a first step, the particulate debris, either host cells or lysed fragments, is removed, for example, by centrifugation or ultrafiltration.

An antibody or antibody fragment) can be purified using, for example, hydroxylapatite chromatography, cation or anion exchange chromatography, or preferably affinity chromatography, using the antigen of interest or protein A or protein G as an affinity ligand. Protein A can be used to purify proteins that include polypeptides are based on human γ1, γ2, or γ4 heavy chains (Lindmark et al., J. Immunol. Meth. 62: 1-13 (1983)). Protein G is recommended for all mouse isotypes and for human γ3 (Guss et al., EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the protein comprises a C_{H} 3 domain, the Bakerbond ABX™ resin (J. T. Baker, Phillipsburg, N.J.) is useful for purification. Other techniques for protein purification such as ethanol precipitation, Reverse Phase HPLC, chromatofocusing, SDS-PAGE, and ammonium sulfate precipitation are also possible depending on the antibody to be recovered.

Chimeric, Humanized, Human Engineered™, Xenomouse® Monoclonal Antibodies.

Chimeric monoclonal antibodies, in which the variable Ig domains of a rodent monoclonal antibody are fused to human constant Ig domains, can be generated using standard procedures known in the art (See Morrison, S. L., et al. (1984) Chimeric Human Antibody Molecules; Mouse Antigen Binding Domains with Human Constant Region Domains, Proc. Natl. Acad. Sci. USA 81, 6841-6855; and, Boulianne, G. L., et al, Nature 312, 643-646. (1984)). A number of techniques have been described for humanizing or modifying antibody sequence to be more human-like, for example, by (1) grafting the non-human complementarity determining regions (CDRs) onto a human framework and constant region (a process referred to in the art as humanizing through “CDR grafting”) or (2) transplanting the entire non-human variable domains, but “cloaking” them with a human-like surface by replacement of surface residues (a process referred to in the art as “veneering”) or (3) modifying selected non-human amino acid residues to be more human, based on each residue's likelihood of participating in antigen-binding or antibody structure and its likelihood for immunogenicity. See, e.g., Jones et al., Nature 321:522 525 (1986); Morrison et al., Proc. Natl. Acad. Sci., U.S.A., 81:6851 6855 (1984); Morrison and 01, Adv. Immunol., 44:65 92 (1988); Verhoeyer et al., Science 239:1534 1536 (1988); Padlan, Molec. Immun. 28:489 498 (1991); Padlan, Molec. Immunol. 31(3):169 217 (1994); and Kettleborough, C. A. et al., Protein Eng. 4(7):773 83 (1991); Co, M. S., et al. (1994), J. Immunol. 152, 2968-2976); Studnicka et al. Protein Engineering 7: 805-814 (1994); each of which is incorporated herein by reference in its entirety.

A number of techniques have been described for humanizing or modifying antibody sequence to be more human-like, for example, by (1) grafting the non-human complementarity determining regions (CDRs) onto a human framework and constant region (a process referred to in the art as humanizing through “CDR grafting”) or (2) transplanting the entire non-human variable domains, but “cloaking” them with a human-like surface by replacement of surface residues (a process referred to in the art as “veneering”) or (3) modifying selected non-human amino acid residues to be more human, based on each residue's likelihood of participating in antigen-binding or antibody structure and its likelihood for immunogenicity. See, e.g., Jones et al., Nature 321:522 525 (1986); Morrison et al., Proc. Natl. Acad. Sci., U.S.A., 81:6851 6855 (1984); Morrison and 01, Adv. Immunol., 44:65 92 (1988); Verhoeyer et al., Science 239:1534 1536 (1988); Padlan, Molec. Immun. 28:489 498 (1991); Padlan, Molec. Immunol. 31(3):169 217 (1994); and Kettleborough, C. A. et al., Protein Eng. 4(7):773 83 (1991); Co, M. S., et al. (1994), J. Immunol. 152, 2968-2976); Studnicka et al. Protein Engineering 7: 805-814 (1994); each of which is incorporated herein by reference in its entirety.

Antibodies can also be produced using transgenic animals that have no endogenous immunoglobulin production and are engineered to contain human immunoglobulin loci. (See, e.g., Mendez et al., Nat. Genet. 15:146-156 (1997)) For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions.

Using a transgenic animal described above, an immune response can be produced to a selected antigenic molecule, and antibody producing cells can be removed from the animal and used to produce hybridomas that secrete human-derived monoclonal antibodies. Immunization protocols, adjuvants, and the like are known in the art, and are used in immunization of, for example, a transgenic mouse as described in WO 96/33735. The monoclonal antibodies can be tested for the ability to inhibit or neutralize the biological activity or physiological effect of the corresponding protein. See also Jakobovits et al., Proc. Natl. Acad. Sci. USA, 90:2551 (1993); Jakobovits et al., Nature, 362:255-258 (1993); Bruggermann et al., Year in Immuno., 7:33 (1993); Mendez et al., Nat. Genet. 15:146-156 (1997); and U.S. Pat. No. 5,591,669, U.S. Pat. No. 5,589,369, U.S. Pat. No. 5,545,807; and U.S Patent Application No. 20020199213. U.S. Patent Application No. and 20030092125 describes methods for biasing the immune response of an animal to the desired epitope. Human antibodies may also be generated by in vitro activated B cells (see U.S. Pat. Nos. 5,567,610 and 5,229,275).

Antibody Production by Phage Display Techniques

The development of technologies for making repertoires of recombinant human antibody genes, and the display of the encoded antibody fragments on the surface of filamentous bacteriophage, has provided another means for generating human-derived antibodies. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference in its entirety. The antibodies produced by phage technology are usually produced as antigen binding fragments, e.g. Fv or Fab fragments, in bacteria and thus lack effector functions. Effector functions can be introduced by one of two strategies: The fragments can be engineered either into complete antibodies for expression in mammalian cells, or into bispecific antibody fragments with a second binding site capable of triggering an effector function.

Typically, the Fd fragment (V_{H}-C_{H}1) and light chain (V_{L}-C_{L}) of antibodies are separately cloned by PCR and recombined randomly in combinatorial phage display libraries, which can then be selected for binding to a particular antigen. The antibody fragments are expressed on the phage surface, and selection of Fv or Fab (and therefore the phage containing the DNA encoding the antibody fragment) by antigen binding is accomplished through several rounds of antigen binding and re-amplification, a procedure termed panning. Antibody fragments specific for the antigen are enriched and finally isolated.

Phage display techniques can also be used in an approach for the humanization of rodent monoclonal antibodies, called “guided selection” (see Jespers, L. S., et al., Bio/Technology 12, 899-903 (1994)). For this, the Fd fragment of the mouse monoclonal antibody can be displayed in combination with a human light chain library, and the resulting hybrid Fab library may then be selected with antigen. The mouse Fd fragment thereby provides a template to guide the selection. Subsequently, the selected human light chains are combined with a human Fd fragment library. Selection of the resulting library yields entirely human Fab.

A variety of procedures have been described for deriving human antibodies from phage-display libraries (See, for example, Hoogenboom et al., J. Mol. Biol., 227:381 (1991); Marks et al., J. Mol. Biol, 222:581-597 (1991); U.S. Pat. Nos. 5,565,332 and 5,573,905; Clackson, T., and Wells, J. A., TIBTECH 12, 173-184 (1994)). In particular, in vitro selection and evolution of antibodies derived from phage display libraries has become a powerful tool (See Burton, D. R., and Barbas III, C. F., Adv. Immunol. 57, 191-280 (1994); and, Winter, G., et al., Annu. Rev. Immunol. 12, 433-455 (1994); U.S. patent application no. 20020004215 and WO92/01047; U.S. patent application no. 20030190317 published Oct. 9, 2003 and U.S. Pat. No. 6,054,287; U.S. Pat. No. 5,877,293. Watkins, “Screening of Phage-Expressed Antibody Libraries by Capture Lift,” Methods in Molecular Biology, Antibody Phage Display: Methods and Protocols 178: 187-193, and U.S. Patent Application Publication No. 20030044772 published Mar. 6, 2003 describes methods for screening phage-expressed antibody libraries or other binding molecules by capture lift, a method involving immobilization of the candidate binding molecules on a solid support.

The invention will be more fully understood by reference to the following examples. These examples are not to be construed in any way as limiting the scope of this invention.

### EXAMPLES

### Example 1

### Backcrossing and Outcrossing

Because of the reported neonatal lethality of Na_{V}1.7 KO animals (heterozygous Nav1.7^{+/-} mice from Deltagen, San Mateo, Calif.: B6.129P2-Scn9 atm1Dgen/J backcrossed at least 8 generations to C57BL/6) due to an apparent failure to feed (Nassar et al., 2004), we outcrossed these animals onto a CD1 background to add vigor to the line, as well as a separate backcross onto a BALB/c line to create a congenic line (first breeding pair received in May 2011). (See, FIG. 1A-B).

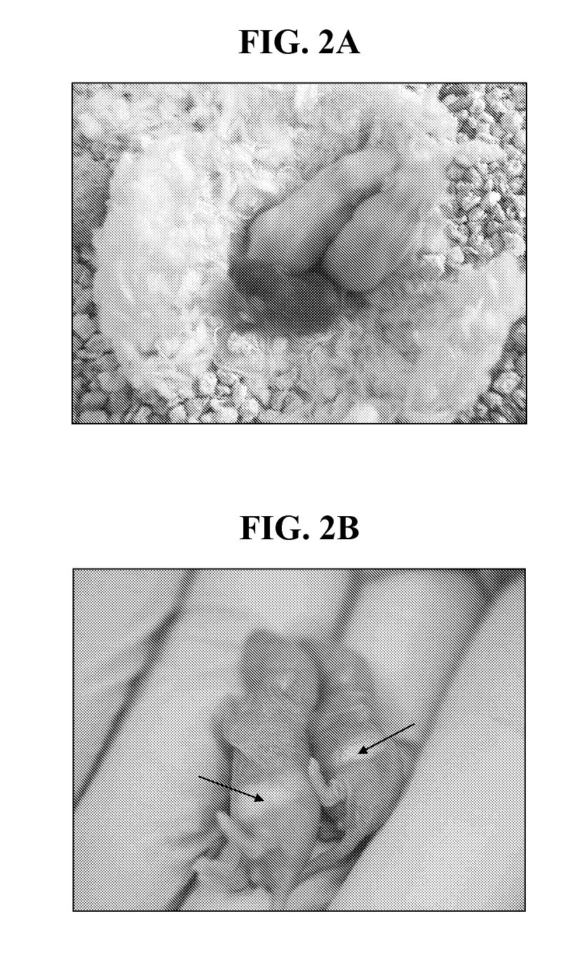

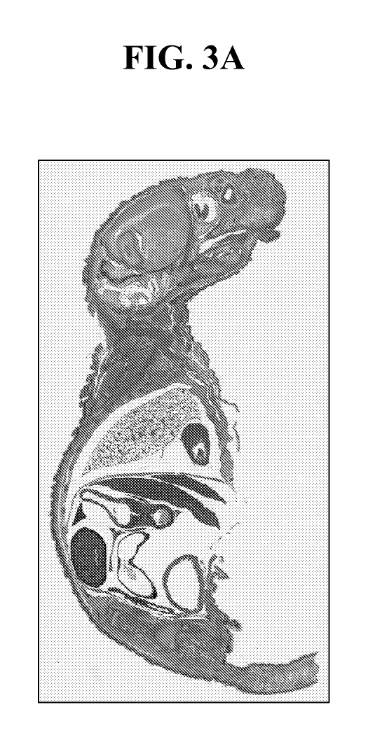

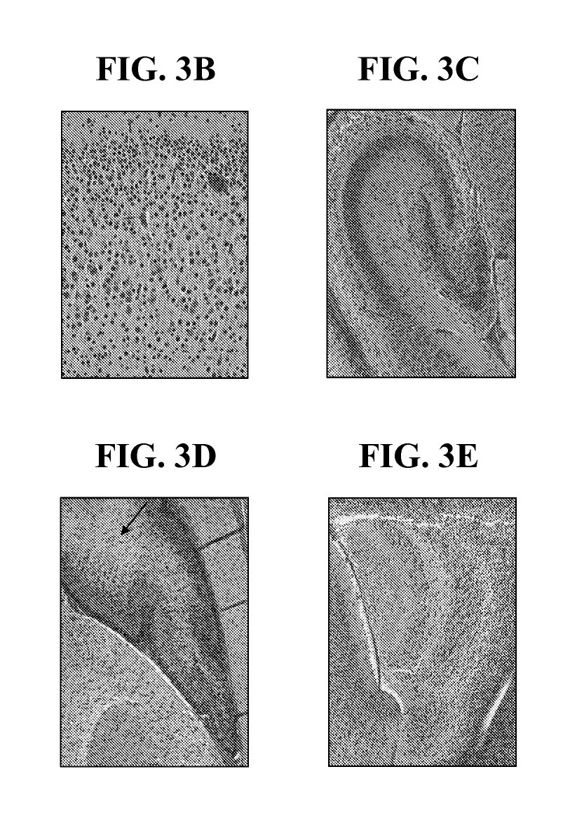

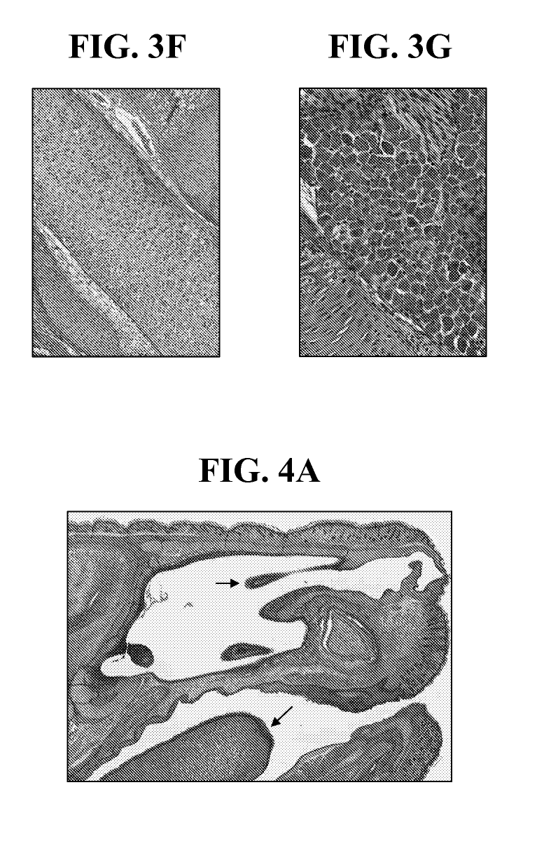

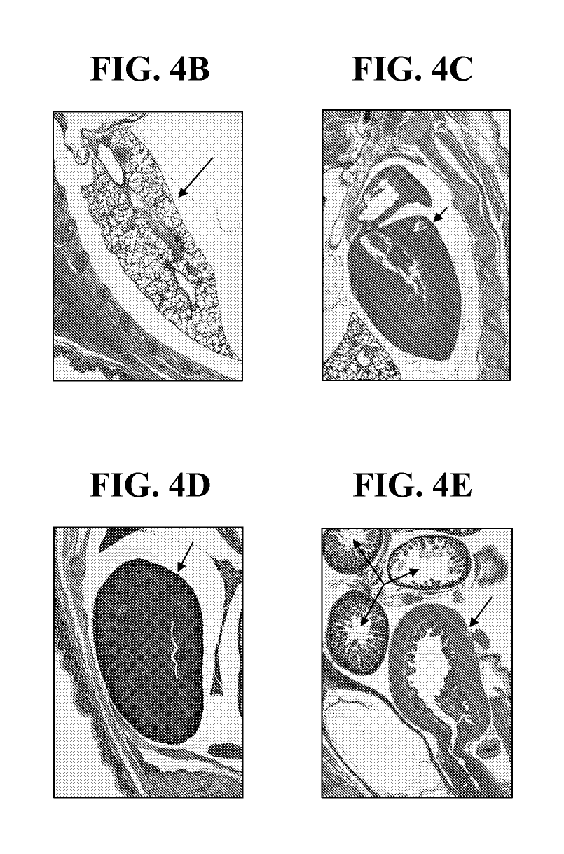

No significant differences were noted in Scn9a-CD1 KO or Scn9a-Balbc KO animals. We refer to these animals as Na_{V}1.7 KO and specify the strains when required. All data displayed herein were collected on single out- and backcross. Further backcrosses (for BALB/c line only) are currently being performed, thus far results obtained were identical on a N4 backcross generation. No further increase in survival was observed. The final outcome of the backcrossing has yet to be determined upon reaching a congenic line at N7 (−99% BALB/c). Na_{V}1.7 KO mice were born roughly at the same size and weight as their litter mates (e.g., FIG. 2B) but differences quickly became apparent within a litter (within 16 hours post-birth; FIG. 2A). The Na_{V}1.7 KO mice did not develop as fast as their litter mates and were a little weaker. Na_{V}1.7 KO pups had a harder time competing for resources and ended up at the bottom of the nest (away from the food source) and eventually died. Because of their obvious capabilities in feeding/suckling milk, we wondered whether placing Na_{V}1.7 KO pups with a foster mother apart from wild type siblings would give them a better chance at survival. Unfortunately, their small size and relative weakness did not allow for proper stimulation of the dams. We quickly noticed a reduction in the milk production (lactation), and neonatal Na_{V}1.7 KO animals died. After a thorough anatomical evaluation of a P4 Na_{V}1.7 KO neonate, we came to the conclusion that these animals were capable of feeding if given the chance. All required organs were in place as depicted in FIG. 3A-G and FIG. 4A-E.

An artificial mouse milk was developed (recipe modified from Auestad et al., 1989 and Yajima et al., 2006; see Example 2 herein).

First, we identified all Na_{V}1.7 KO candidates in a litter according to their weight 24 hours after birth. Roughly the smallest quarter of the litter was identified as potential knockouts, corresponding to the expected Mendelian ratio of 25% (for Het×Het breeding pairs). Larger “control” animals were removed from the nest according to these equations:

[number of candidate KO]+2 controls (if KO number is ≦2)

[number of candidate KO]+3 controls (if KO number is ≧3)

The presence of some control animals allowed for sufficient lactation stimulation of the dam and their low numbers reduced internal litter competition. Control animals were a mixture of wild type and heterozygous of both gender, as determined by standard polymerase-chain-reaction (PCR).

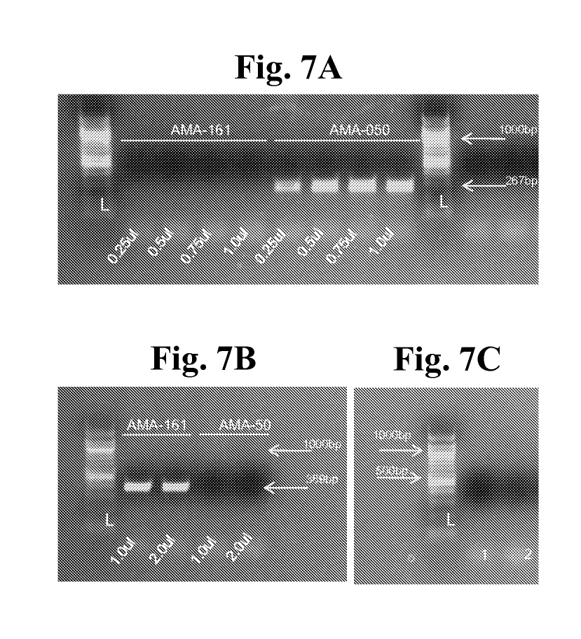

Briefly, the following primers were used to genotype all animals in the colony: Forward Scn9a: 5′-AGA CTC TGC GTG CTG CTG GCA AAA AC-3′ (SEQ ID NO:1); Forward Neomycin: 5′-GGG CCA GCT CAT TCC TCC CAC TCA T-3′ (SEQ ID NO:3) and Reverse Scn9a: 5′-CGT GGA AAG ACC TTT GTC CCA CCT G-3′ (SEQ ID NO:2). These primers gave rise to an endogenous (E) band of 267 base pairs (primer Forward Scn9a+primer Reverse Scn9a); (see bands in FIG. 7A) or a targeted (T) band of 389 base pairs (primer Forward Neomycin+primer Reverse Scn9a); see bands in FIG. 7B). The expected genotyping patterns were as follows:

WT (+/+) animals=endogenous (E) band only

HET (+/−) animal=endogenous (E)+targeted (T) bands

KO (−/−) animal=targeted (T) band only.

Since the KO phenotype is determined by the “absence” of an endogenous (E) band, we first tested a range of DNA concentrations (25 ng to 100 ng) to ensure that we had enough intact DNA for the PCR reaction to generate a product. As shown in FIG. 7A, as little as 25 ng of DNA was sufficient to generate a visible PCR product in a wild type animal (AMA-50). In the presence of identical DNA concentrations, no PCR product was present in the KO animal (AMA-161), suggesting that this animal does carry an intact copy of the endogenous Scn9a gene. In FIG. 7B, the presence of a PCR product (band) for the Targeted primer pairs confirms that (i) Na_{V}1.7 KO DNA (AMA-161) was intact, of good quality and that (ii) animal AMA-161 carries the targeted Neo gene and thus qualifies as a Na_{V}1.7 KO mouse. In FIG. 7C, controls of PCR reaction samples did not contain DNA, and thus did not generate a PCR product.

Animals that were confirmed as Na_{V}1.7 KO were reconfirmed in two ways. Firstly, they underwent a battery of behavioral tests in which the unique phenotype that they display (lack of thermal pain sensation and presence of anosmia; see, Example 4 herein) further confirmed the genotype. Secondly, all animals were re-genotyped by the same PCR method (described above) upon euthanization.

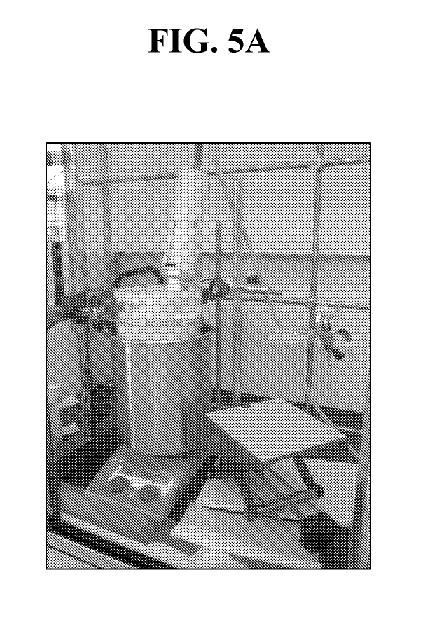

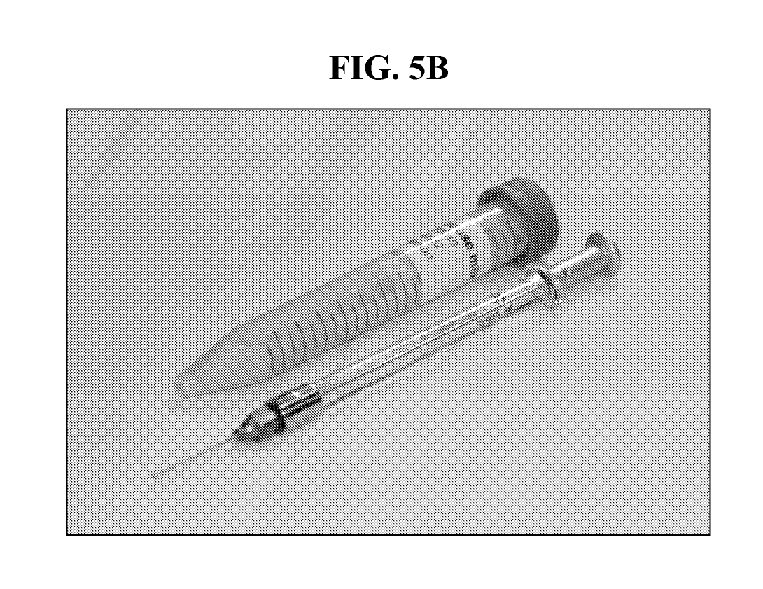

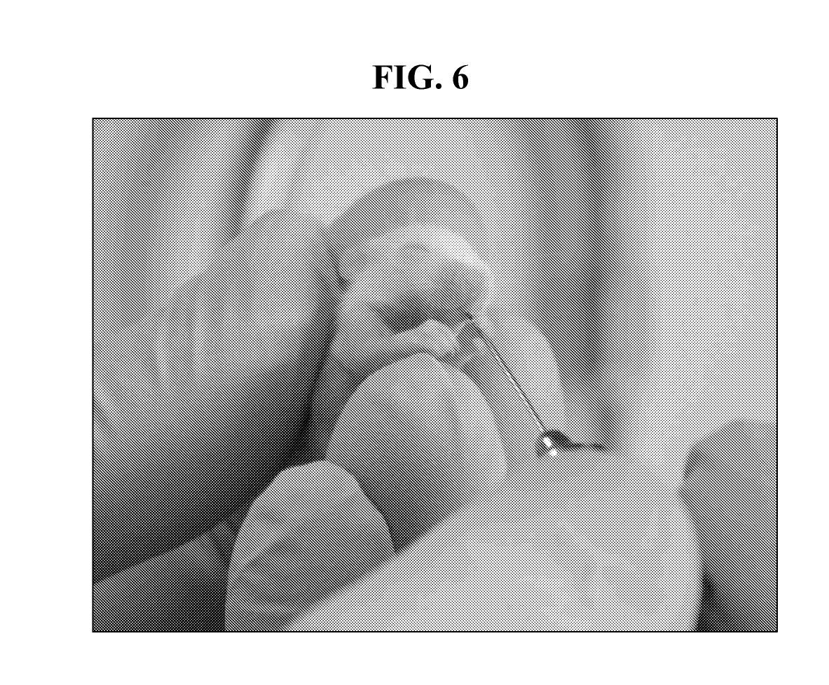

Na_{V}1.7 KO candidates were hand fed (see, syringe in FIG. 5B and feeding in FIG. 6) three times a day/7 days a week for duration of 14 to 21 days. When teeth erupted, soft supplement food was made available for all neonate mice as an extra source of calories. A total of 28 Na_{V}1.7 KO animals were generated over an 11 month period: 24 on a CD1 background and 4 on a BALB/c background. The difference in numbers between the two backgrounds was a reflection of the size of the colony and not of the difficulty or outcome of the breeding strategies. We found that animals behave in very similar ways on either background. Eighteen (18) animals remained alive as of Dec. 1, 2010 (see “status” column of Table 2). Eight (8) were lost for health-related issues, and two (2) animals on study (immunization assay) had to be euthanized following health complications due to the immunization. Their deaths could not be attributed to a particular phenotype. Animals of both genders were present in the expected ratios (˜50%) for each strain. Confirmed genotyped animals were 54 days to 7.5 months old as of Dec. 1, 2010. Table 2 (below) shows a list of Na_{V}1.7 KO mouse individuals. Upon reaching adulthood, Na_{V}1.7 KO animals were able to mate. Na_{V}1.7 KO animals, both males (Table 2A) and females (Table 2B), were crossed with opposite gender heterozygous (HET) animals from the same background. Both males and females were able to reproduce. Males were particularly slow and inefficient at the task, 18 separate mating events leading to the generation of one litter (Table 2A). In some individual cases, post-mortem analysis of the uro-genital apparatus was conducted to determine sperm count and investigate any fertility issues. Both fertile and infertile males were seen. For example, AMA-161 was analyzed and judged infertile (Sertoli cells and spermatogonia were present but mature spermatozoids were absent, but a similar phenotype was also seen in some control animals), while AMA-380 was fertile. The low success observed in Na_{V}1.7 KO male breeding might be related to the anosmia (lack of sense of smell) phenotype observed in Na_{V}1.7 KO mice. Na_{V}1.7 KO males, may not be able to pick-up the pheromones of females placed in their cages. Females of both strains were reproductively successful (3 litters out of 4 mating events) (Table 2B). All females studied could carry to term, drop the litter and lactate/feed the pups. A sense of smell would not be required for the females for breeding purposes, as the males placed in their cages were Na_{V}1.7 HETs that showed no signs of anosmia that might interfere with pairing.

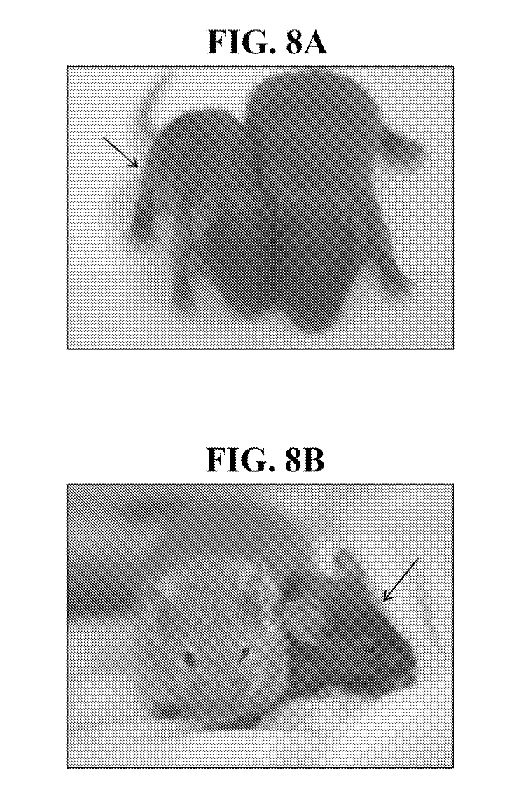

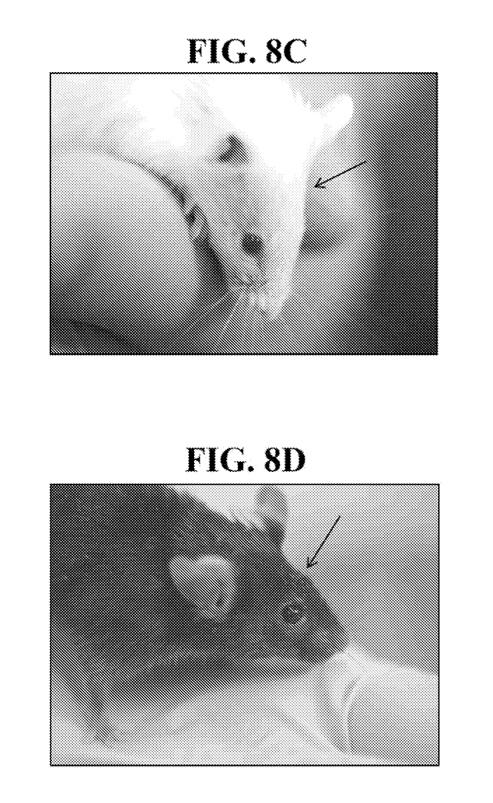

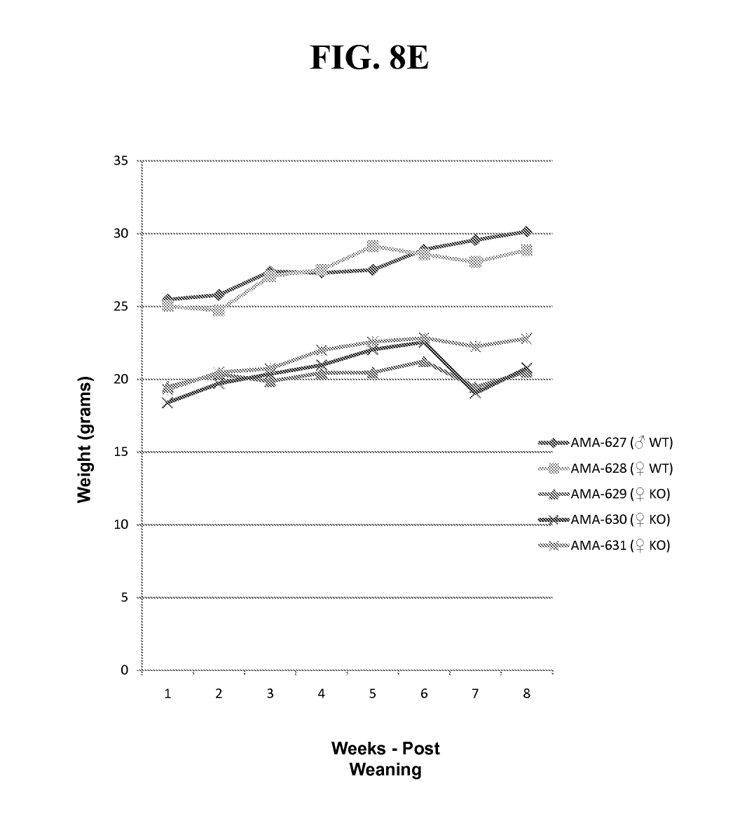

FIG. 8A-E illustrates the external phenotype of Na_{V}1.7 KO mice. The external phenotype was normal, as shown in FIG. 8A-D, except for a noticeable difference in size (see smaller Na_{V}1.7 KO mice indicated by arrows in FIG. 8A-D). Their eyes were open, their teeth erupted, and their coats were well developed. They were mobile in the cages approximately at the same time as their littermates. As early as 16 hours after birth, Na_{V}1.7 KO animals were seen to be smaller than their littermates. FIG. 8E shows a size comparison over 8 weeks post-weaning. When compared to their post weanling wild type littermates, Na_{V}1.7 KO animals were ˜25% smaller. This size difference could be due to a calorie restriction encountered during development, hence the requirement for hand feeding as a compensatory source (but not as a full replacement).

Clinical Events Observed in Na_{V}1.7 KO Mice.

The Na_{V}1.7 KO colony was monitored over a span of about 1 year. Aside from the intensive neonatal care these animals require, a few recurrent post-weaning health issues were noticed. It is not clear whether these were related to the genetic disorder or due to intensive manipulation throughout the neonatal period. Dermatitis was observed in ˜40% of the Na_{V}1.7 KO population, in both females (8 cases) and males (3 cases). This clinical event was successfully resolved with an optimized treatment combining a once weekly Baytril (Western Medical Supply, Inc., Arcadia, Calif.; cat. #2269) treatment, followed by a twice daily topical application of Triple Antibiotic Ointment (Butler Schein Animal Health, Dublin, Ohio; cat. #031087). When dermatitis appeared in the vicinity of the eyes, a Topical Triple Ophthalmic Ointment (Webster Veterinary, Sterling, Mass.; cat. #07-805-4502) was used instead. For the mild cases, Baytril treatment alone was sufficient to resolve the issue with a full recovery. Based on the data collected from the behavioral itch assay (see, Example 4 herein), Diphenhydramine was not administered to the Nav1.7 KO mice as they are insensitive to histamine-induced itch. Enlarged bladders were observed in ˜25% of the Na_{V}1.7 KO mouse colony. This was more preponderant in males (5 cases) than females (2 cases). Treatment consisted of a manual pressure gently applied onto the enlarged bladder area to allow the urine to be released. In addition, subcutaneous injections of NaCl fluids were combined when animals showed signs of dehydration. Penile prolapse was reported in a few males and was resolved by manually placing the organ back in its cavity combined with Topical Triple Antibiotic, when redness was observed. All animals showing clinical signs were placed on Teklad Diamond Soft Bedding (Harlan Laboratories, Indianapolis, Ind.; cat. #7089) to minimize abrasive contact with standard bedding and/or to monitor urine production when blockages were detected.

TABLE 2
List of Na_{V}1.7 KO mouse individuals.

| Mouse # | DOB | strain | genotype | gender | color | |
|---|---|---|---|---|---|---|
| | | | | | | age |
| | | | | | | (days)* |
| AMA-161 | Dec. 15, 2009 | Scn9a-CD1 | KO | M | black | n/a |
| AMA-318 | Feb. 26, 2010 | Scn9a-CD1 | KO | F | black | n/a |
| AMA-380 | Mar. 10, 2010 | Scn9a-CD1 | KO | M | agouti | n/a |
| AMA-457 | Mar. 26, 2010 | Scn9a-BalbC | KO | F | agouti | n/a |
| AMA-473 | Mar. 30, 2010 | Scn9a-CD1 | KO | M | black | n/a |
| AMA-507 | Apr. 13, 2010 | Scn9a-CD1 | KO | M | white | 227 |
| AMA-508 | Apr. 13, 2010 | Scn9a-CD1 | KO | M | black | 227 |
| AMA-553 | May 3, 2010 | Scn9a-CD1 | KO | F | white | 207 |
| AMA-579 | May 10, 2010 | Scn9a-CD1 | KO | M | black | n/a |
| AMA-582 | May 10, 2010 | Scn9a-CD1 | KO | F | white | n/a |
| AMA-595 | May 18, 2010 | Scn9a-CD1 | KO | F | white | 192 |
| AMA-598 | May 18, 2010 | Scn9a-CD1 | KO | F | white | 192 |
| AMA-599 | May 18, 2010 | Scn9a-CD1 | KO | F | black | 106 |
| AMA-607 | May 25, 2010 | Scn9a-BalbC | KO | M | black | 185 |
| AMA-608 | May 25, 2010 | Scn9a-BalbC | KO | F | agouti | 185 |
| AMA-623 | Jun. 7, 2010 | Scn9a-BalbC | KO | M | agouti | n/a |
| AMA-629 | Jun. 8, 2010 | Scn9a-CD1 | KO | F | white | 172 |
| AMA-630 | Jun. 8, 2010 | Scn9a-CD1 | KO | F | white | 172 |
| AMA-631 | Jun. 8, 2010 | Scn9a-CD1 | KO | F | white | 172 |
| AMA-642 | Jun. 27, 2010 | Scn9a-CD1 | KO | M | black | 153 |
| AMA-669 | Jul. 13, 2010 | Scn9a-CD1 | KO | F | white | 137 |
| AMA-671 | Jul. 19, 2010 | Scn9a-CD1 | KO | M | black | 131 |
| AMA-681 | Jul. 19, 2010 | Scn9a-CD1 | KO | M | white | 131 |
| AMA-691 | Aug. 2, 2010 | Scn9a-CD1 | KO | M | agouti | n/a |
| AMA-694 | Aug. 2, 2010 | Scn9a-CD1 | KO | F | white | n/a |
| AMA-754 | Sep. 13, 2010 | Scn9a-CD1 | KO | F | white | 77 |
| AMA-784 | Oct. 5, 2010 | Scn9a-CD1 | KO | F | white | 56 |
| AMA-787 | Oct. 6, 2010 | Scn9a-CD1 | KO | F | white | 55 |
| AMA-876 | Nov. 23, 2010 | Scn9a-BalbC | KO | F | white | 7 |
| AMA-892 | Nov. 29, 2010 | Scn9a-CD1 | KO | F | white | 1 |
| | | | | | | age |
| | | | | | | (days)** |
| AMA-1982 | Aug. 01, 2011 | Scn9a-CD1 | KO | F | black | n/a |
| AMA-2025 | Oct. 17, 2011 | Scn9a-CD1 | KO | F | white | n/a |
| AMA-2026 | Oct. 17, 2011 | Scn9a-CD1 | KO | F | black | n/a |
| AMA-2048 | Oct. 31, 2011 | Scn9a-CD1 | KO | M | agouti | n/a |
| AMA-2076 | Nov. 6, 2011 | Scn9a-CD1 | KO | F | black | 73 |
| AMA-2081 | Nov. 7, 2011 | Scn9a-CD1 | KO | F | white | 72 |
| AMA-2125 | Nov. 28, 2011 | Scn9a-CD1 | KO | M | black | 51 |
| AMA-2117 | Nov. 29, 2011 | Scn9a-CD1 | KO | M | black | 50 |
| AMA-2118 | Nov. 29, 2011 | Scn9a-CD1 | KO | F | black | 50 |
| AMA-2119 | Nov. 29, 2011 | Scn9a-CD1 | KO | F | white | 50 |
| AMA-2178 | Dec. 5, 2011 | Scn9a-CD1 | KO | F | white | 44 |
| AMA-2173 | Dec. 6, 2011 | Scn9a-CD1 | KO | M | agouti | 43 |
| AMA-2175 | Dec. 6, 2011 | Scn9a-CD1 | KO | M | agouti | 43 |

*Ages are as of Nov. 30, 2010.
“n/a” indicates euthanized.
**Ages are as of Jan. 18, 2012.

TABLE 2A
Breeding Na_{V}1.7^{-/-} males to a Na_{V}1.7^{+/-} females.

| | | Number of | | |
|---|---|---|---|---|
| | | mating | | |
| Males | Strain | events | Plug | litter |
| AMA-161 | Scn9a-CD1 | 6 | 0 | 0 |
| AMA-380 | Scn9a-CD1 | 6 | 1 | 1 |
| AMA-473 | Scn9a-CD1 | 4 | 0 | 0 |
| AMA-507 | Scn9a-CD1 | 1 | 1 | 0 |
| AMA-508 | Scn9a-CD1 | 1 | 0 | 0 |

TABLE 2B
Breeding Na_{V}1.7^{-/-} females to a Na_{V}1.7^{+/-} males.

| | | Number of | | |
|---|---|---|---|---|
| Females | Strain | mating events | Plug | litter |
| AMA-457 | Scn9a-CD1 | 2 | 1 | 1 |
| AMA-509 | Scn9a-BalbC | 1 | 1 | 1 |
| AMA-553 | Scn9a-CD1 | 1 | 1 | 1 |

### Example 2

### Preparation of Artificial Mouse Milk (AMA)

The procedure and composition of the artificial mouse milk prepared was modeled closely after that described by Yajima and co-workers and by Auestad and co-workers. (Yajima, M, et al., A Chemically Derived Milk Substitute that is Compatible with Mouse Milk for Artificial Rearing of Mouse Pups” Exp. Amin. 55(4), 391-397, (2006); Auestad, N, et al., Milk-substitutes comparable to rat's milk; their preparation, composition and impact on development and metabolism in the artificially reared rat, Br. J. Nutr. 61: 495-518 (1989)).

Reagents and Procedure.

Reagents used, including source, amounts used, solvents and amounts used, and miscellaneous comments are listed in Table 3 (below).

Procedure.

Step 1: Sodium hydroxide was weighed and transferred to a 10-L beaker. Distilled Water (1.3 L) was added and an overhead stirrer was put in place. Potassium hydroxide was weighed and added to this solution, as were L-serine, L-cystine and L-tryptophan. The solution was heated in a water bath to 58° C. If convenient, this solution or mixture can be transferred into bottles and stored in the refrigerator for up to five days before further use. Next, casein was slowly added to the warm solution and the mixture was heated to 71° C. for 90 minutes. This mixture was transferred to a 4-L glass beaker and heated to 50° C. An overhead stirrer was put in place and the mixture was heated to boiling (FIG. 5A). Step 2: Glycerophosphate calcium, magnesium chloride hexahydrate and calcium chloride were weighted, dissolved in 200 mL of distilled water and homogenized for 20 minutes. This mixture was slowly added to the casein mixture with continuous stirring. Step 3: Calcium carbonate and calcium citrate tetrahydrate were weighed, added to 100 mL of distilled water, homogenized for 1 minute and slowly added to the casein mixture. Step 4: Sodium phosphate dibasic heptahydrate and potassium phosphate monobasic were weighed, dissolved in 50 mL of distilled water and slowly added to the casein solution. Step 5: Lactose monohydrate was weighed and homogenized in 220 mL of distilled water before addition to the casein mixture. Step 6: Iron sulfate heptahydrate and citric acid were weighed and dissolved in 5 mL of distilled water and added to the casein mixture. Step 7: Manganese sulfate hydrate, cupric sulfate pentahydrate and zinc sulfate heptahydrate were weighed and dissolved in 5 mL of distilled water before being added to the casein solution. Step 9: Sodium floride and potassium iodide were weighed and dissolved in 5 mL distilled water and added to the casein mixture. Steps 8 and 10: Whey protein was weighed and homogenized in 600 mL of distilled water. To this mixture were added L-carnitine, alpha-picolinic acid HCl, ethanolamine and taurine dissolved in 10 mL distilled water.

Steps 11 and 12: A mixture of palm oil, coconut oil, corn oil, MCT oil, soy oil and cholesterol was heated to 60° C. on a hot plate. Choline dihydrogen citrate and vitamin mixture were weighed and suspended in 70 mL of distilled water. Sodium hydroxide (5 N) was added. This mixture was slowly added to the casein mixture. The oil mixture was added to the casein mixture and the volume brought up to 4 L with distilled water. The mixture was transferred into bottles and stored in the refrigerator until use within 3 days.

The stored artificial milk was removed from the refrigerator and transferred into a 4-L beaker suspended in a water bath and heated to boiling. The homogenizer tip was sterilized by boiling in water for 15 minutes. Step 13: The artificial milk mixture was homogenized as; vitamin K1, vitamin A palmitate and DL-tocopheryl acetate were weighed and added during homogenization. The artificial milk was kept in the boiling water bath with constant homogenization while it was aliquotted into sterile 15-mL Eppendorff tubes (FIG. 5B). The artificial milk was stored at −80° C. until used.

TABLE 3
Reagents used, including source, lot or batch numbers,
amounts used, solvents and amounts used.

| Step | Reagent | Source | Amount | | Solvent |
|---|---|---|---|---|---|
| 1 | Sodium | Fisher | 2.48 | g | Added to |
| | hydroxide | | | | 1.3 L water |
| 1 | Potassium | EMD | 6.86 | g | |
| | hydroxide | | | | |
| 1 | L-Serine | MP | 1.15 | g | |
| 1 | L-Cystine | Fluka | 0.90 | g | |
| 1 | L-Tryptophan | MP | 1.08 | g | |
| 1 | Casein | Sigma | 266.2 | g | |
| 2 | Glycerophosphate | MP | 32.0 | g | |
| | calcium salt | | | | |
| 2 | Magnesium | MP | 6.4 | g | |
| | chloride | | | | |
| | hexahydrate | | | | |
| 2 | Calcium chloride | Fisher | 4.4 | g | Dissolve in |
| | dehydrate | | | | 200 mL |
| | | | | | water |
| 3 | Calcium | Sigma | 10 | g | |
| | carbonate | | | | |
| 3 | Calcium citrate | Alfa Aesar | 4.8 | g | Dissolve in |
| | tetrahydrate | | | | 100 mL |
| | | | | | water |
| 4 | Sodium | J. T. Baker | 3.2 | g | |
| | phosphate dibasic | | | | |
| | heptahydrate | | | | |
| 4 | Potassium | Mallinckrodt | 0.33 | g | Dissolve in |
| | phosophate | | | | 50 mL |
| | monobasic | | | | water |
| 5 | Lactose | Fisher | 74.05 | g | Homogenize |
| | monohydrate | | | | in 220 mL |
| | | | | | water |
| 6 | Iron sulfate | Amgen | 0.92 | g | |
| | heptahydrate | barcode | | | |
| 6 | Citric acid | J. T. Baker | 0.02 | g | Dissolve in |
| | monohydrate | | | | 5 mL |
| | | | | | water |
| 7 | Manganese | Alfa aesar | 3 | mg | |
| | sulfate hydrate | | | | |
| 7 | Cupric sulfat | Fluka | 61 | mg | |
| | pentahydrate | | | | |
| 7 | Zinc sulfate | Acros | 242 | mg | Dissolve in |
| | heptahydrate | | | | 5 mL |
| | | | | | water |
| 8 | Whey protein | Sigma | 160 | g | Homogenize |
| | | | | | in 600 ml |
| | | | | | water |
| 9 | Sodium fluoride | Paratronic | 6 | mg | |
| 9 | Potassium iodide | Paratronic | 11 | mg | Dissolve in |
| | | | | | 5 mL |
| | | | | | water |
| 10 | L-carnitine | Creosalus | 158 | mg | |
| 10 | Alpha-piconilinic | MP | 86 | mg | |
| | acid HCl | | | | |
| 10 | Ethanolamine | MP | 130 | mg | |
| 10 | Taurine | MP | 602 | mg | Dissolve in |
| | | | | | 10 mL |
| | | | | | water |
| 11 | Palm oil | Spectrum | 195.64 | g | |
| 11 | Coconut oil | Spectrum | 160.77 | g | |
| 11 | Corn oil | Sigma | 63.88 | g | |
| 11 | MCT oil | Nestle | 95.73 | g | |
| | | Nutrition | | | |
| 11 | Soy oil | Spectrum | 127.65 | g | |
| 11 | Cholesterol | Calbiochem | 1.7 | mg | Mix |
| 12 | Choline | Aldrich | 5.7 | g | |
| | dihydroen citrate | | | | |
| 12 | Vitamin mixture | Harlan | 19.98 | g | Add 70 mL |
| | | | | | water |
| 12 | 5N NaOH | | 1.82 | mL | |
| 13 | Vitamin K1 | Alfa aesar | 83.6 | mg | |
| 13 | Vitamin A | MP | 5.20 | mg | |
| | palmitate | | | | |
| 13 | DL-tocopheryl | Acros | 120.7 | mg | |
| | acetate | | | | |

### Example 3

### Genotyping

The following primers were used to genotype all animals in the colony:

Forward Scn9a 5′ AGA CTC TGC GTG CTG CTG GCA AAA AC 3′ (SEQ ID NO:1); Reverse Scn9a 5′ CGT GGA AAG ACC TTT GTC CCA CCT G 3′ (SEQ ID NO:2) and Forward Neomycin 5′ GGG CCA GCT CAT TCC TCC CAC TCA T 3′(SEQ ID NO:3). These primers gave rise to an endogenous band of 267 base pairs (Forward Scn9a+Reverse Scn9a) or a targeted band of 389 base pairs (Forward Neomycin+Reverse Scn9a). PCR cycling conditions were as follows:

(1) 95° C. for 7 minutes, followed by 35× cycles of (2)-(4) below, followed by (5)-(6):

(2) 96° C. for 10 seconds;

(3) 60° C. for 30 seconds;

(4) 72° C. for 1.5 minutes,

(5) 72° C. for 7 minutes;

(6) cool to 4° C.

The genotyped patterns were as follows:

WT or +/+=endogenous (E) band only

HET or +/−=endogenous (E)+targeted (T) bands

KO or −/−=targeted (T) band only

DNA concentrations ranging between ˜25 ng up to 1 μg were originally tested for each sample. Endogenous band was never present in any of the AMA-161 samples (or any of the other Nav1.7 KO samples), the first confirmed live Na_{V}1.7 KO animal (on a CD1 background; FIG. 7A-C). Animal AMA-50 is a confirmed wild type (WT). Controls containing no DNA were blank, as expected.

### Example 4

### Pain Testing

Thermal Tests.

The thermal paw stimulator is an apparatus that allows the investigator to deliver a discrete thermal stimulus (radiant heat) to a specific area (e.g., the paw). Animals were housed in a testing chamber on top of a glass surface heated to 25 C. At the onset of the test, a thermal beam coupled with a timer is switched on under the hind paw. Movement of the animals' paw in response to the stimulus terminates the stimulus and served as the endpoint of the test. A maximal cut-off time of 20 sec was used to prevent tissue damage. Animals were typically tested two times with an inter-trial interval of at least five minutes. If the first two measurements were not consistent, one or two more trials were used to clarify the animal's true thermal threshold. Latency to remove the paw from the thermal source was recorded as the endpoint.

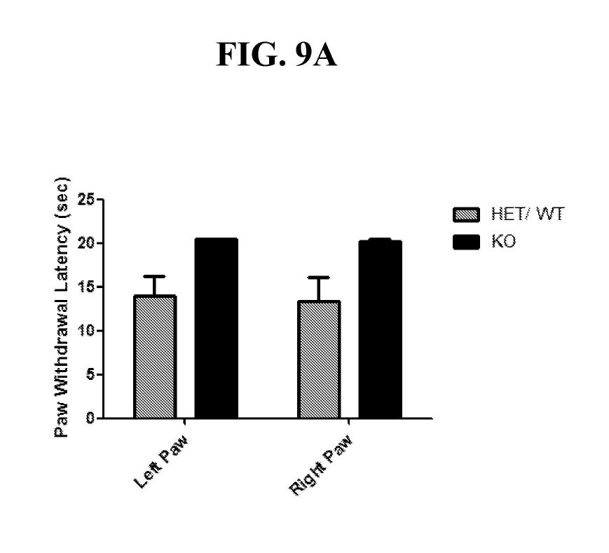

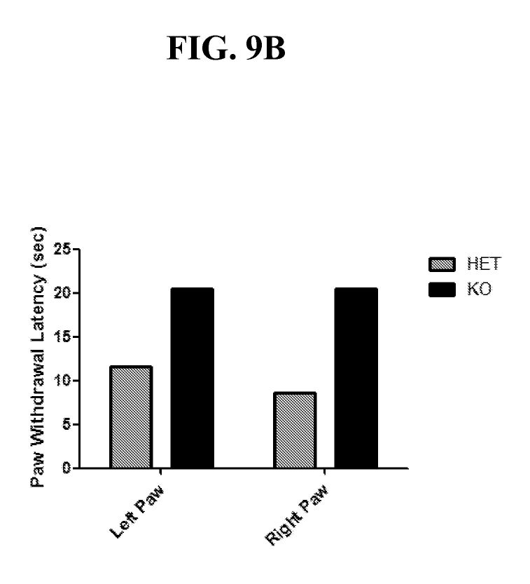

FIG. 9A shows Na_{V}1.7 KO mouse strain Scn9a-CD1 apparently had a slight response to thermal challenge (Hargreaves Apparatus) in the right paw (n=3), and did not react in the left paw (n=5). FIG. 9B shows that Na_{V}1.7 KO mouse strain Scn9a-BalbC did not respond in either paw (n=1). No differences were seen between WT and HET (FIG. 9A-B), they all reacted normally and withdrew their paws within 10 to 15 sec of the application of the thermal stimulus. The vast majority of the Na_{V}1.7 KO animals tolerated the thermal stimulus until the maximal cut-off time was reached (20 sec).

The hot plate apparatus has a controllable heated surface set to predetermined temperatures. A mouse was then placed on the apparatus and the response to heat was monitored. Responses include paw lifting, paw licking, flinching and/or jumping. A maximal time limit was always utilized and varies depending upon stimulus intensity (i.e., temperature). For acute tests, maximal time allowed on the apparatus was based on temperature and was as follows: 48° C.=60 sec; 50° C.=40 sec; 52.5° C.=30 sec; 55° C.=20 sec. For repeated trials, there was an inter-trial interval of at least ten minutes to allow the paw to fully recover from the test. At the start of the test, the animals were placed into the testing chamber and a timer was started. Animals were removed from the apparatus immediately following a response or at the maximal cut-off time, whichever occurred first. Latency to first response was recorded as the endpoint.

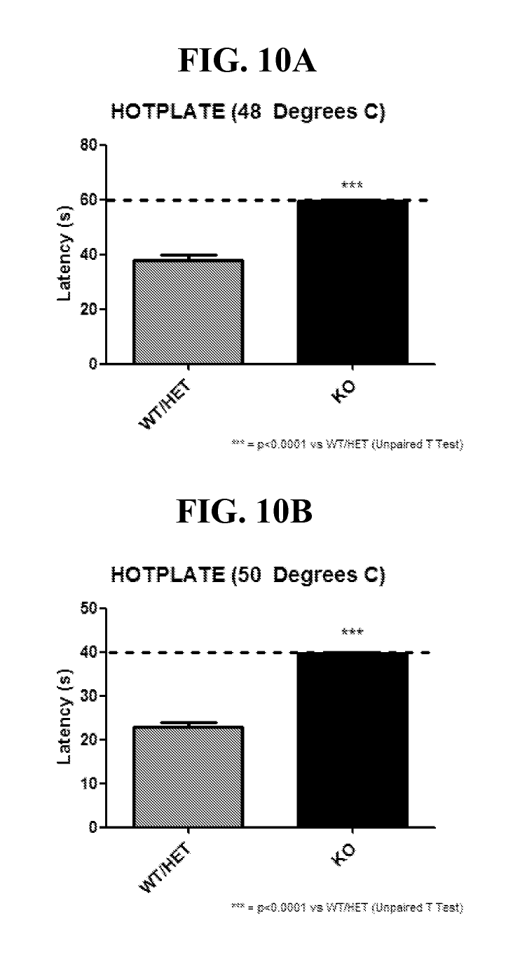

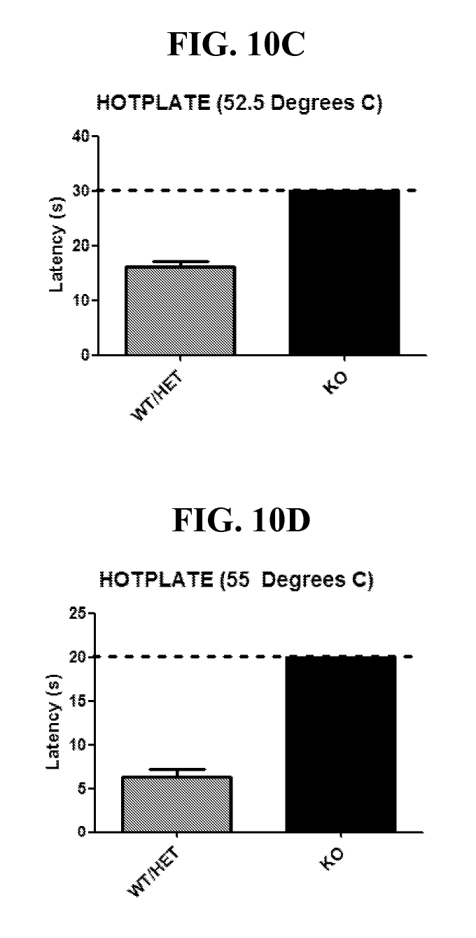

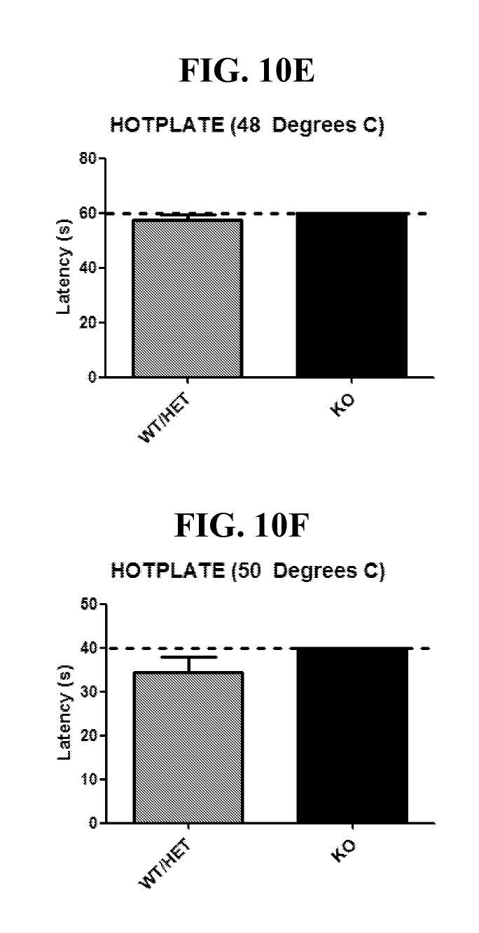

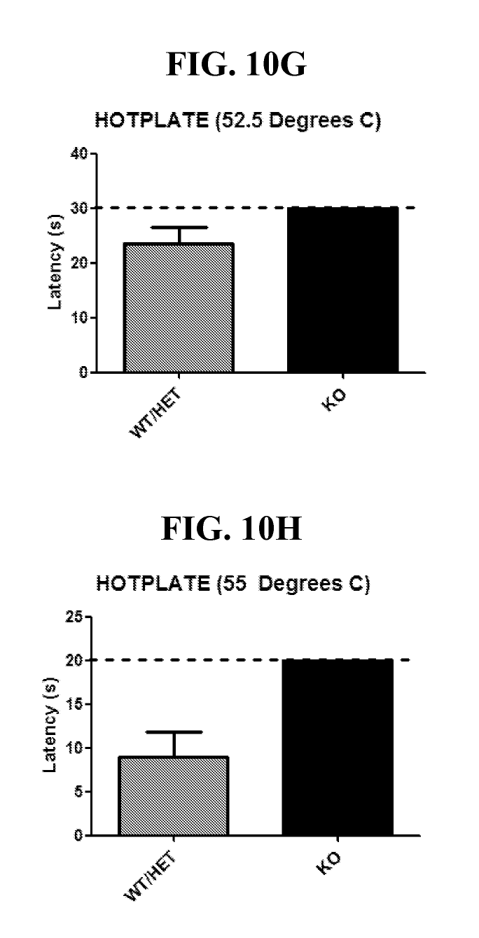

FIG. 10A-H shows that both Na_{V}1.7 KO mouse strains were insensitive to thermal pain, tested at four different temperatures (48, 50, 52.5, and 55° C.). Scn9a-CD1 Na_{V}1.7^{-/-} KO mice (FIG. 10A-D; n=14) and Scn9a-BalbC Na_{V}1.7^{-/-} KO mice (FIG. 10E-H; n=4) were insensitive to thermal pain, even at the highest testable temperatures of 55° C., at which they had to be removed from the hot plate at the set cut off time (20 sec) to avoid tissue damage.

Tactile Allodynia-Von Frey Test.

Von Frey filaments are used to assess mechanical sensitivity in rodents. Mice are placed on a wire mesh floor, enclosed in an individual testing chamber and allowed to acclimate until calm. Calibrated filaments of various bending forces were then applied to the paw of a mouse to measure the response to a non-noxious tactile (e.g., touch) stimulus. The pattern of responses and non-responses to the series of filaments determined the animal's mechanical threshold. This threshold was used as the endpoint of the assay.

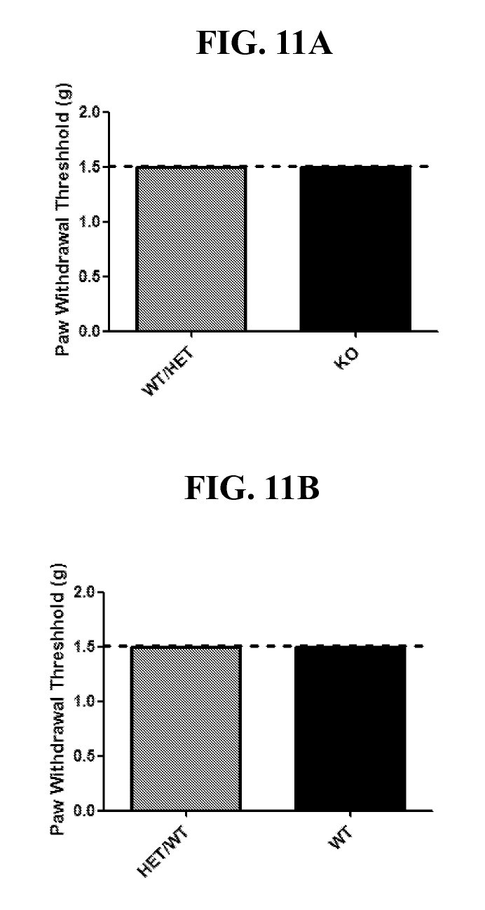

FIG. 11A-B shows that Na_{V}1.7 KO mouse strains Scn9a-CD1 (FIG. 11A; n=16) and Scn9a-BalbC (FIG. 11B; n=4) reacted in a similar fashion as their wild type/heterozygous control littermates to a Von Frey allodynia challenge. All mice tested reached cut-off threshold of 1.5 g. Na_{V}1.7 KO animals seemed to perceive mechanical pressure normally, consistent with reported observations of Na_{V}1.7 deletion in humans.

Anosmia Testing.

The Buried Food Test has been developed in order to assess whether a mouse's sense of olfaction is intact. Briefly, an animal was trained to identify a pina colada scented food pellet by placing one pellet into the home cage. Each animal was checked the following morning to ensure the pellet was eaten and recognized as food. The food was placed in the cage three times prior to the test day, one per day. Animals were then food deprived for 18 hours prior to the test day. On test day, the mouse was transferred to a standard mouse cage with 3 cm of clean bedding and allowed to acclimate for five minutes. After five minutes, the mouse was transferred to another clean cage with a food pellet buried 1 cm under the bedding in a random corner. The mouse was then reintroduced to the cage. Latency to find and start eating the food was recorded and served as the endpoint of the study. If the animal was not able to find the food after 15 minutes, the test was stopped.

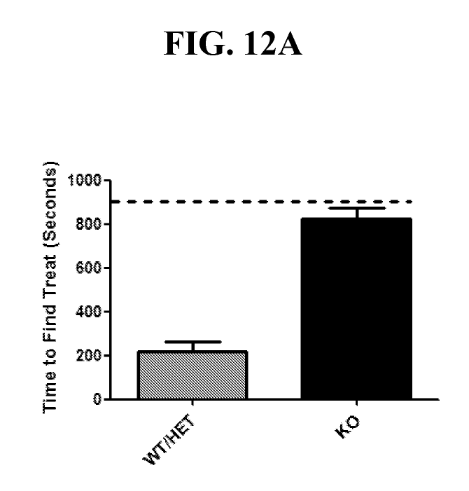

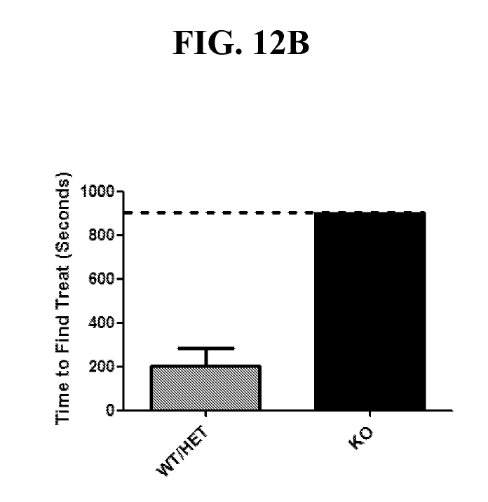

FIG. 12A-B shows that food-deprived Na_{V}1.7 KO mouse strains Scn9a-CD1 (FIG. 12A; n=14) and Scn9a-BalbC (FIG. 12B; n=4) had difficulties (FIG. 12A) or failed (FIG. 12B) in locating a hidden scented food pellet during the allocated time of 15 minutes, compared to age-matched/sex-matched control (WT/HET) littermates who retrieved the pellets within the first 200 seconds of the assay.

Itch Testing.

On test day, all animals were conditioned to observation chambers for 30 minutes prior to the irritant injection. An injection of 150 μg of histamine diphosphate was administered intradermally in a volume of 100 μL on the back of the animal between the shoulder blades. This area was shaved the day prior to the test in order to aid in injection placement. Intradermal injections were performed while the mouse was gently restrained by hand. After histamine injections, animals were placed in observation chambers and bouts of scratching were counted for up to 40 minutes. The number of itch bouts was recorded as the endpoint.

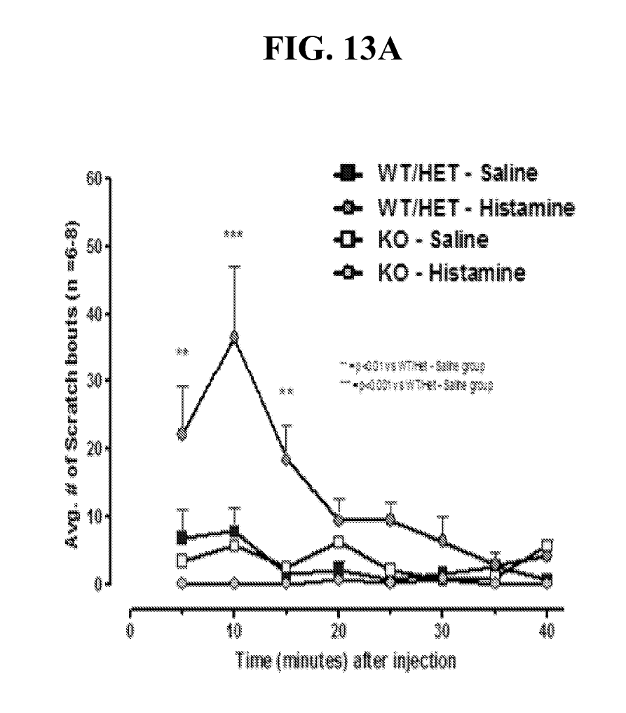

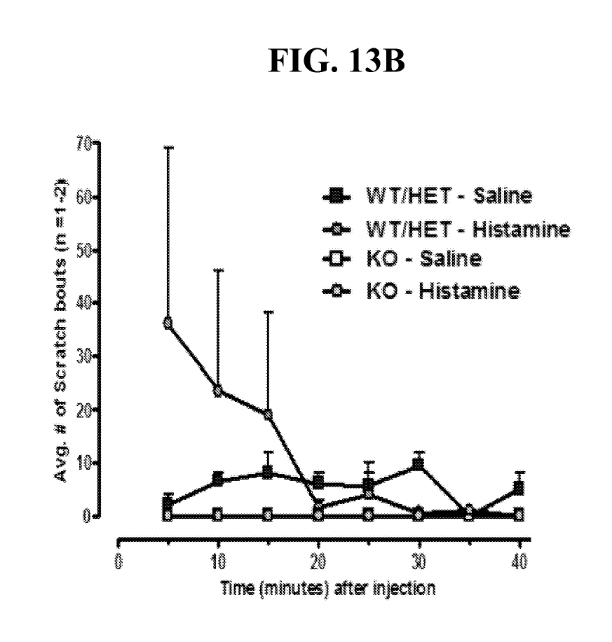

FIG. 13A-B shows that unlike their wild type/heterozygous littermates, Na_{V}1.7 KO mouse strains Scn9a-CD1 (FIG. 13A; n=12) and Scn9a-BalbC (FIG. 13B; n=3) were insensitive to histamine (see diamonds) and showed very few (FIG. 13A) or no (FIG. 13B) scratching bouts following histamine injection. The average number of scratch bouts performed by the Na_{V}1.7 KO mice were within the range of those performed following saline injection in wild type/heterozygous control littermates (inverted triangles). In particular, the wild type/heterozygous animals scratched heavily (upright triangles) during the first 10 minutes of the assay while the Na_{V}1.7 KO (diamonds) were not responsive.

### Example 5

### Use of Na_{V}1.7 Knockout Mice to Identify a Biomarker for Na_{V}1.7 Inhibitors

On-Target Biochemical Challenge for Na_{V}1.7 Inhibitors.

Voltage-gated sodium channels are primary determinants of neuronal excitability, and accordingly are potential targets for novel therapies directed against neurological disorders of hyperexcitability, including pain. Clinical evidence from human genetic disorders shows that among nine sodium channels, the Na_{V}1.7 subtype encoded by the SCN9A gene is critical for the transmission of pain, making it an attractive point for development of a targeted inhibitor. There are, however, key challenges to developing a therapy against Na_{V}1.7. A first hurdle is finding a molecule with sufficient selectivity for Na_{V}1.7 as opposed to other sodium channel subtypes; the literature contains reports of compounds that are selective for individual Nav subtypes. (Jarvis, M F et al., A-803467, a potent and selective Nav1.8 sodium channel blocker, attenuates neuropathic and inflammatory pain in the rat, Proc Natl Acad Sci USA. 104(20):8520-25 (2007); Beaudoin, S et al., Sulfonamide Derivatives, WO 2010/079443 A1). Key additional steps are to optimize the in vivo drug-like properties of a candidate molecule, including mode of delivery, nonspecific plasma protein binding, half life and other pharmacokinetic properties. Following this a candidate compound may be tested in preclinical species for behavioral efficacy in animal models of disease, including pain. Subsequent toxicology testing defines therapeutic window and guides dosing.

In all these steps, it is critical to know whether the candidate molecule actually engages the target—a difficult step to take from just efficacy and exposure. For example, sometimes very large plasma or brain exposures do not translate into target engagement; equilibrium plasma protein binding determined from in vitro dialysis or centrifugation experiments may not necessarily predict the true active amount of the candidate molecule; and engagement of many proteins, individually or separately, by a candidate molecule can produce efficacy (real or spurious) in animal models of disease, particularly pain. A critical challenge is to know via an on-target biomarker in an in vivo system that a candidate compound has reached its intended target and exerted functional effects. This is important in establishing the most accurate therapeutic window. In clinical development of a candidate molecule this challenge is even more important. For example, a negative clinical trial without target engagement indicates further trials, whereas a negative clinical trial in which the tested therapeutic did engage its biomarker generally ends further development.

Nav1.7 is a sodium ion channel activated by voltage, and not by any chemical neurotransmitter or ligand that might be used as the basis of a biomarker. As far as known, its sole function is to produce the electrical spike of a neuron, and not to initiate or modulate any signal transduction pathways. This makes monitoring inhibition of Na_{V}1.7 function by assaying downstream biochemical effects very difficult. Accordingly, there are no known biomarkers for Na_{V}1.7, particularly not one known to be specific for Na_{V}1.7 amongst all nine voltage-gated sodium channels.

We have developed an in vivo assay that serves as a biomarker for Na_{V}1.7 inhibitors, using nonspecific modulators of voltage-gated sodium channels and the inventive Na_{V}1.7 global KO mice described herein. The sodium channel modulator veratridine produced a quantifiable, robust, dose-dependent, and reproducible licking and paw-flinching behavior in mice and in rats that developed with a characteristic time course. These pain-like behaviors were inhibited somewhat by some (e.g., morphine, duloxetine) but not all (e.g., gabapentin) existing medications for pain, and were inhibited fully by a nonspecific sodium channel blocker (mexiletine). Painful flinches were observed with two additional chemically distinct sodium channel modulators, deltamethrin (structure II below) and grayanotoxin III (“GRAY3”; structure III below), further evidence that the assay reflects sodium channel function and not an effect unique to veratridine.

An assay was developed to measure veratridine-induced flinching in rats. Importantly, veratridine had no effect when tested on mice missing Na_{V}1.7 (global knockout Na_{V}1.7), showing that the effects of veratridine are mediated entirely by Na_{V}1.7. Accordingly, this aspect of the invention represents an on-target and on-mechanism biochemical challenge with which to test experimental Na_{V}1.7 blockers in mammalian subjects, including mouse, rats, rabbits, ferrets, dogs, non-human primates, or humans, e.g., by skin application analogous to capsaicin studies that serve as biochemical challenge markers for experimental inhibitors of VR1. (Chizh, B A et al., The effects of the TRPV1 antagonist SB-705498 on TRPV1 receptor-mediated activity and inflammatory hyperalgesia in humans, Pain 132(1-2):132-141 (2007); Gavva N R, Bannon A W, et al., Repeated administration of vanilloid receptor TRPV1 antagonists attenuates hyperthermia elicited by TRPV1 blockade, J Pharmacol Exp Ther 323:128-137 (2007)). This aspect of the invention represents a major advantage for selecting appropriate doses for clinical trials and interpreting clinical study results, as well as a tool for best sodium channel drug discovery.

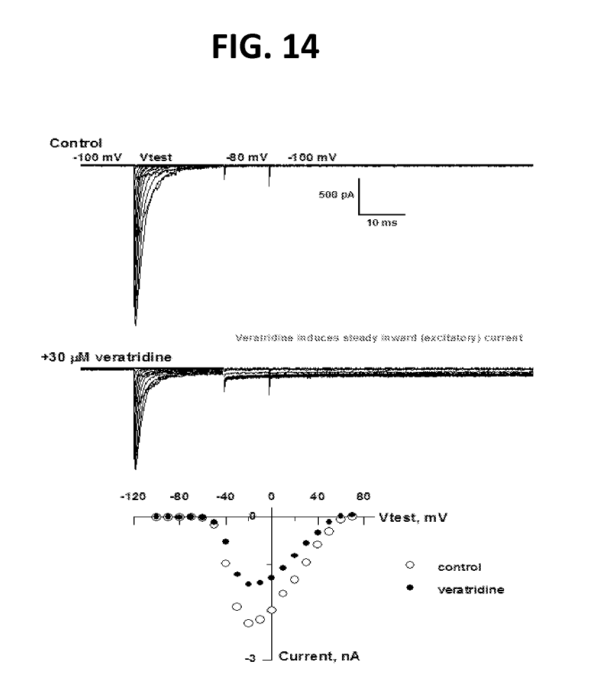

FIG. 14 shows in vitro modulation of Na_{V}1.7 by veratridine (4α,9-Epoxy-3β-veratroyloxy-5β-cevan-4β,12,14,16β,17,20-hexaol; chemical structure shown below in I). Currents shown in FIG. 14 are unsubtracted. Addition of 30 μM veratridine lowered the peak currents, as described, and produced a steady, long-lasting inward current upon return to negative membrane potential. This second effect would be expected to produce continuous influx of positively charged sodium ions into a neuron expressing Na_{V}1.7, producing spiking of the neuron subsequently perceived as pain.

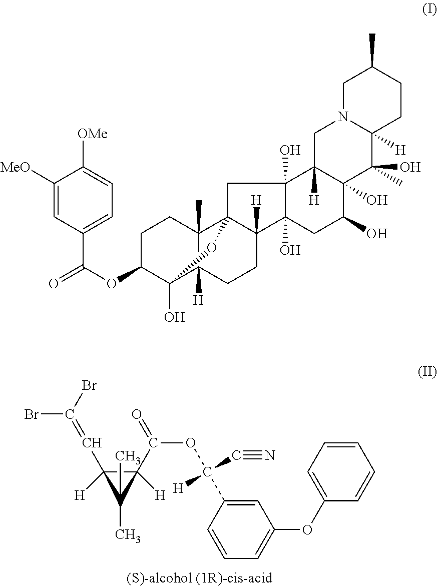

(III)

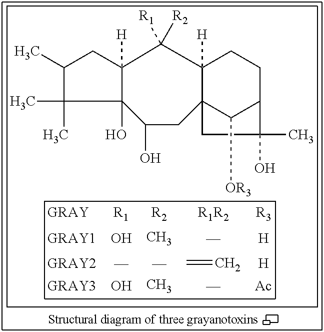

Currents through hNa_{V}1.7 stably expressed in HEK 293 cells were evoked by a family of depolarizing voltage pulses at 10-mV intervals from a holding voltage of −100 mV, using the whole-cell configuration of the patch-clamp technique.

Whole Cell Patch Clamp Electrophysiology.

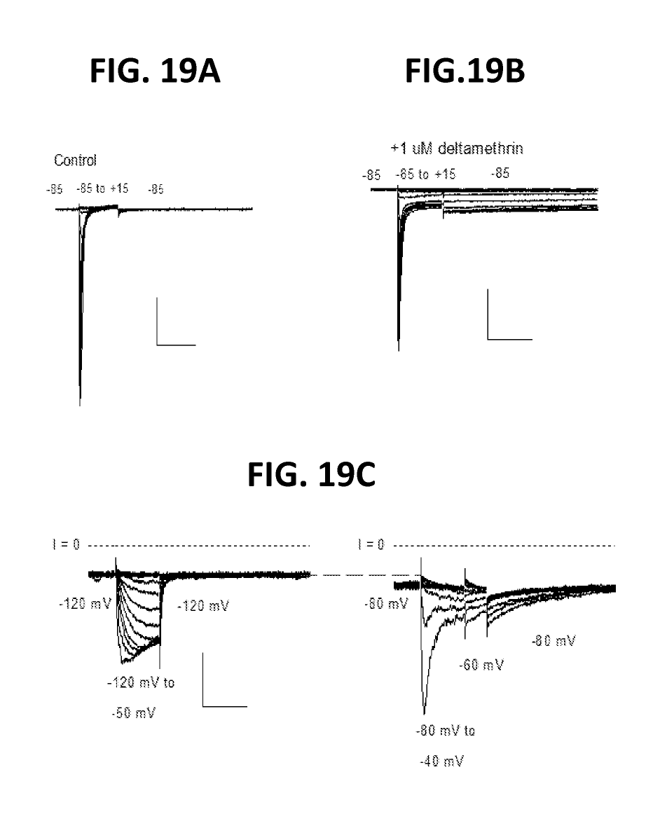

Cells were voltage clamped using the whole cell patch clamp configuration at room temperature (˜22° C.). Pipette resistances were between 1.5 and 2.0 M. Whole cell capacitance and series resistance were uncompensated. Currents were filtered (4-pole Bessel) at 5 kHz during acquisition and digitized at 20 kHz using pClamp9.2. Cells were lifted off the culture dish and positioned directly in front of a micropipette connected to a solution exchange manifold for compound perfusion. To monitor sodium currents, 10 ms pulses to −10 mV were delivered every 5 seconds, and currents were recorded before and after external compound addition in the case of deltamethrin and veratridine, or in the case of grayanotoxin III with grayanotoxin III included in the intracellular (pipette) solution. External solution consisted of: 140 mM NaCl, 5.0 mM KCl, 2.0 mM CaCl₂, 1.0 mM MgCl₂, 10 mM HEPES, and 11 mM Glucose, pH 7.4 by NaOH. Internal solution consisted of: 62.5 mM CsCl, 75 mM CsF, 2.5 mM MgCl₂, 5 mM EGTA, and 10 mM HEPES, pH 7.25 by CsOH. Results are shown in FIG. 19A-C, illustrating that deltamethrin and grayantoxin activated hNa_{V}1.7, and in FIG. 14 illustrating that veratridine activated hNa_{V}1.7, in response to a family of step depolarizations as indicated.

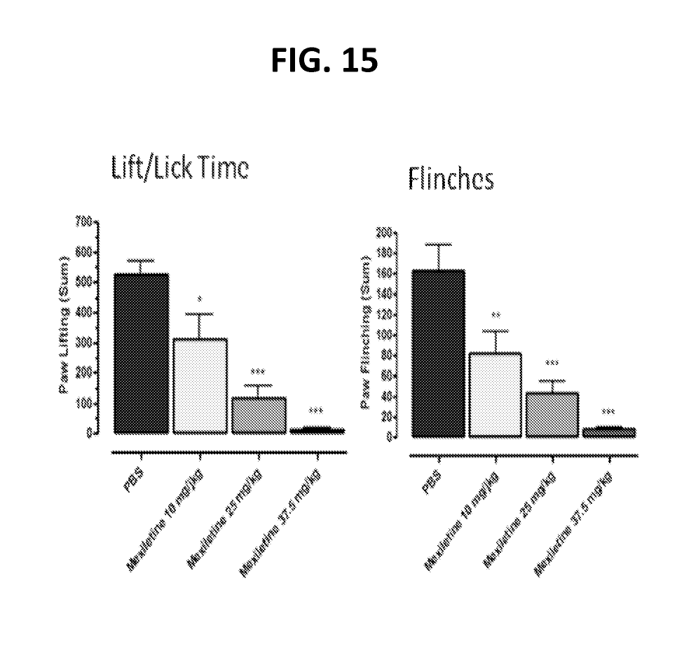

Veratridine injected into the paw of rats (Sprague-Dawley, male unless otherwise specified, aged 8 to 10 weeks) caused two separate pain-related behaviors; animals lifted and licked the paw, and animals also flinched the paw (FIG. 15). Veratridine was injected intra-plantar at 30 micrograms. Higher doses produced variable neurological side effects, including “wet-dog” shakes, in addition to pain-related behaviors and were not studied further. Lifting and flinching behaviors were recorded for 20 minutes following veratridine injection, and both behaviors were prevented in a dose-dependent manner by the sodium channel blocker mexiletine, administered orally (p.o.) one hour before veratridine injection.

Known preclinical and clinical analgesics were tested to see if they could prevent veratridine-induced behaviors. Table 4 shows that duloxetine and morphine both reversed the flinching behavior, but not the paw-lifting behavior. This suggests that the veratridine-evoked response does reflect pain, but may have a pharmacologically distinct component not controlled by morphine or duloxetine. Additional analgesics gabapentin and the anti-inflammatory naproxen did not reduce either lifting or flinching, suggesting that veratridine-induced pain either goes by a different pathway or is too intense to be alleviated by these comparatively weak drugs. “Compound 52” is an aminotriazine Na_{V}1.7 blocker optimized for in vivo use, described in Bregman et al., Identification of a potent, state-dependent inhibitor of Nav1.7 with oral efficacy in the formalin model of persistent pain, J. Med. Chem. 54(13):4427-45 (Jul. 14, 2011; epub Jun. 2, 2011). It is effective in the formalin model of persistent pain. Diazepam is an anti-anxiolytic that is not an effective analgesic preclinically or clinically, and it was ineffective in the veratridine model, again consistent with the observed flinching behavior representing pain.

TABLE 4
Effects of analgesic compounds on pain testing in male rats (strain).
Compound 52 is described in Bregman et al., Identification of a
potent, state-dependent inhibitor of Nav1.7 with oral efficacy in
the formalin model of persistent pain, J. Med. Chem. 54(13):
4427-45 (Jul. 14, 2011; epub Jun. 2, 2011).

| | | Significant | Significant |
|---|---|---|---|
| | Significant Reversal | Reversal of | Reversal of |
| Compound Tested | of Lifting | Flinching | Edema |
| Mexiletine | Yes | Yes | Yes |
| Duloxetine | No | Yes | No |
| Morphine | No | Yes | No |
| Gabapentin | No | No | No |
| Naproxen | No | No | No |
| Diazepam | No | No | No |
| Compound 52 | Yes | Yes | Yes |

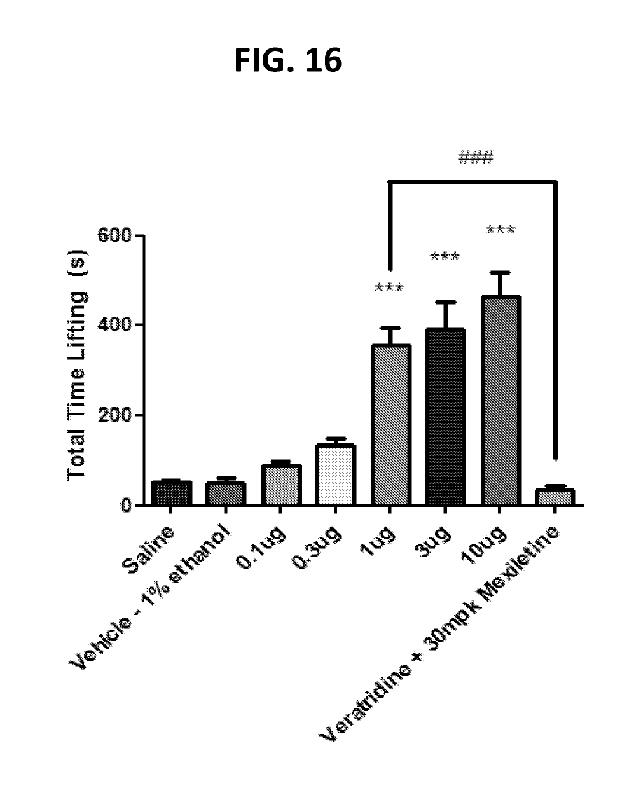

Veratridine caused equivalent lifting behavior injected into male mice (CD1 strain, obtained from Harlan Laboratories, Inc., Indianapolis, Ind.), weights ranging from 35 grams to 45 grams), again in a dose-dependent manner. Shown in FIG. 16 are total lifting time recorded for 30 minutes following i.pl. injection of the indicated dose of veratridine in 1% ethanol in phosphate-buffered saline. Effects of veratridine were blocked by the nonspecific sodium channel inhibitor mexiletine, dosed 30 mg/kg i.p. 30 minutes before challenge with 1 microgram veratridine.

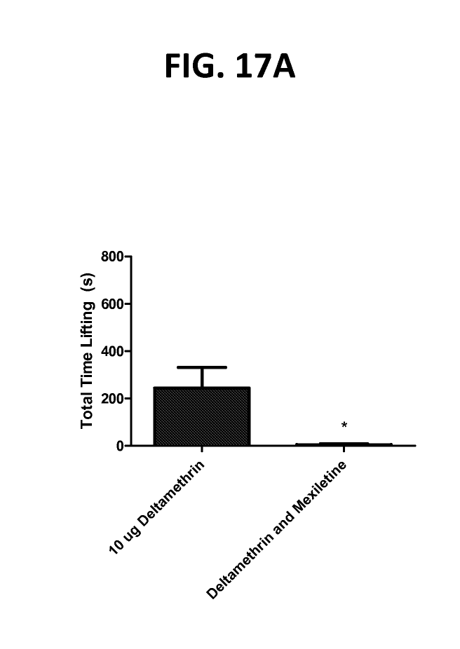

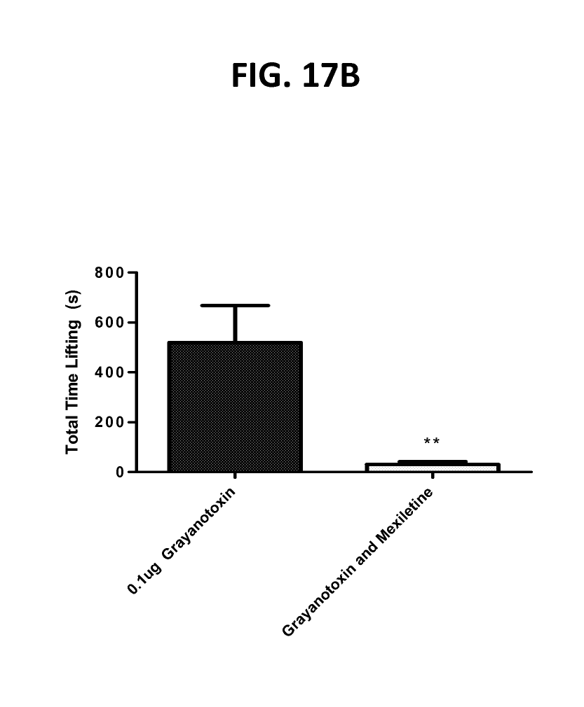

Two other sodium channel modulators were injected separately into male mice (CD1 strain, obtained from Harlan), and each produced a lifting/licking response. Shown in FIG. 17A-B are total flinches in response to a 10 microgram suspension dose of deltamethrin (FIG. 17A; n=6) or a 0.1-microgram dose of grayanotoxin III (FIG. 17B; n=6) in solution with 1% ethanol in phosphate-buffered saline. Pre-administration of mexiletine at 30 mg/kg i.p. in saline solution prevented the lifting/licking response to either deltamethrin or to grayanotoxin III. Weights of the mice studied with deltamethrin ranged from 30 grams to 45 grams, and weights of the mice studied with grayanotoxin III ranged from 30 grams to 45 grams. Results support that the painful behaviors associated with veratridine are not just specific to veratridine, but rather to activation of one or more sodium channels, since three unrelated molecules sharing a functional effect on sodium channels produced the same result.

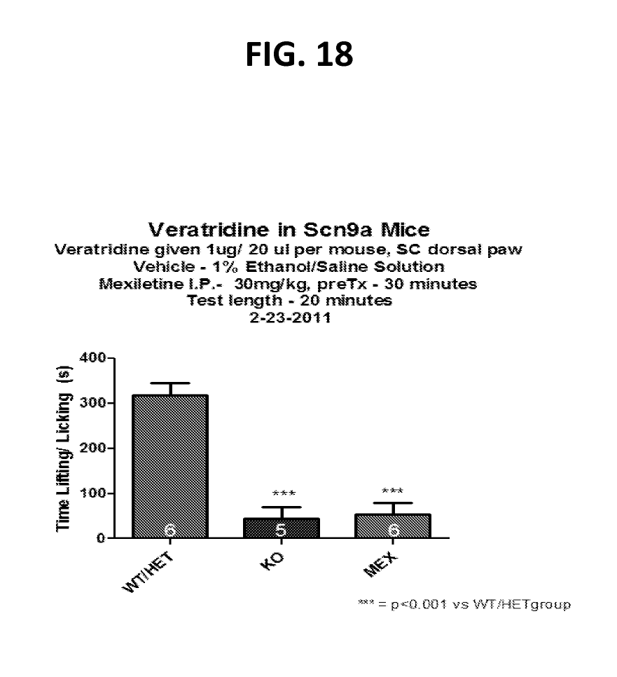

Activation of one, some, or all of the nine sodium channel isoforms might produce painful behaviors. The best biomarker, however, is one that detects specific inhibitors of Na_{V}1.7 (Scn9a). To this end, one microgram of veratridine was injected into the paws of adult global knockout Na_{V}1.7 mice (n=5) and wild type heterozygote littermates (n=6). The knockout mice are missing Na_{V}1.7 since birth; contrary to published literature, Nav1.7 removal does not necessarily result in neonatal lethality. Whereas veratridine injection produced a robust flinching response in the wild type heterozygote mice, the same amount and volume of veratridine produced no response in Na_{V}1.7 knockouts (FIG. 18). With the exception of absent sense of smell and of pain, healthy Na_{V}1.7 knockout mice like those used in this experiment show no apparent defects, including in open-field tests of overall movement. The small amount of “lifting” time remaining in the knockout column can be ascribed to the normal amount of time a mouse would spend lifting or licking its paw over the course of thirty minutes. All behavioral experiments were done with the observer blinded as to the mouse genotype or treatment. Veratridine-induced lifting/licking behavior in wildtype heterozygote mice also was prevented by pharmacological administration of mexiletine.

The results described in this Example 5 show that sodium channel activators produce a robust and quantifiable painful response, that this response is mediated solely via activation of Nav1.7, and that this response is sensitive to pharmacological inhibition of sodium channels. Accordingly, the inventive veratridine biomarker assay described herein represents an on-target biochemical challenge assay specific for Na_{V}1.7.

### Example 6

### Use of Na_{V}1.7 Knockout Mice to Generate Anti-Nav1.7 Antibodies

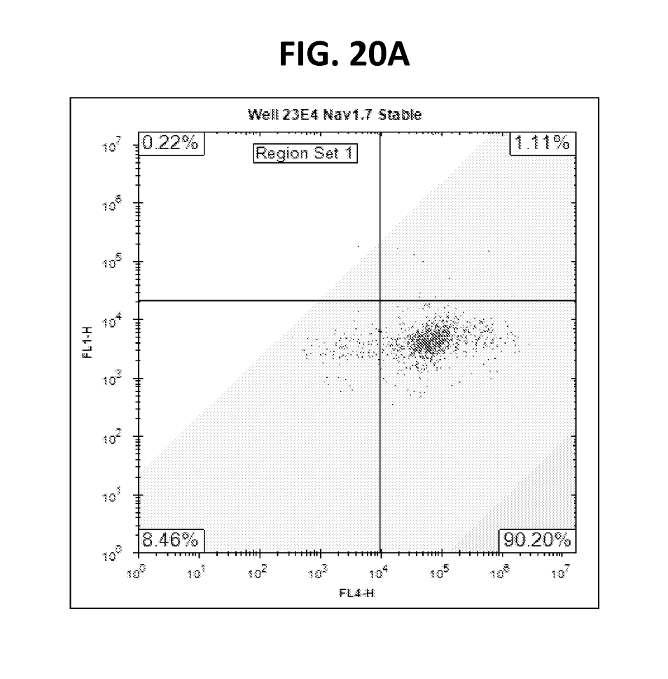

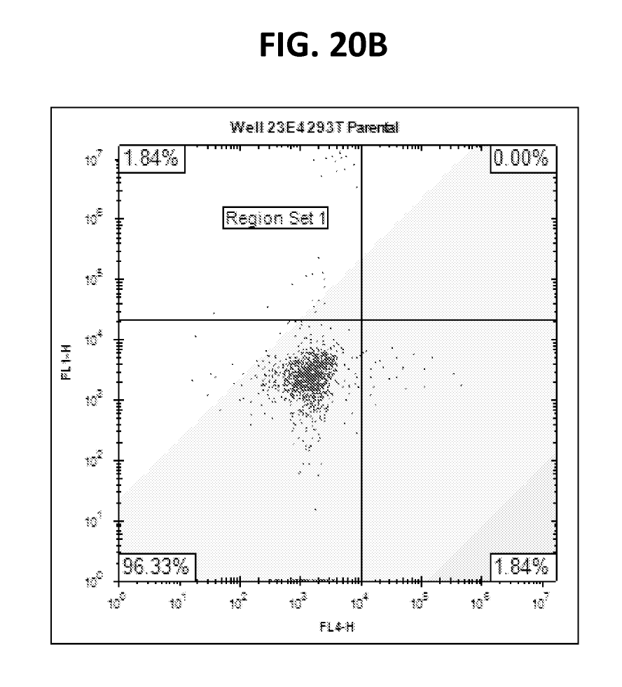

FIG. 20A-B demonstrates antibody generation by Na_{V}1.7 knockout mice of the present invention. Na_{V}1.7 knockout mice were immunized with cells expressing human Na_{V}1.7. Antibody-secreting hybridoma cells were prepared from mouse spleen fusions by standard procedures, hybridoma cells were cultured, and supernatants were isolated from each individual well of the hybridoma cells.

FIG. 20A-B show tests of a representative hybridoma supernatant for the presence of anti-Na_{V}1.7 antibodies, using flow cytometry. HEK 293 cells, either parental 293 cells or cells stably expressing human Na_{V}1.7, were incubated with the test supernatant and with a fluorescent-tagged anti-mouse secondary antibody. Fluorescence emission was measured from each cell with flow cytometry fluorescence-activated cell sorting (FACS) gating on a healthy cell population. To determine antibody binding, 2×10⁵ human Na_{V}1.7-expressing cells were incubated with a 1:20 dilution of supernatant containing IgG for 1 hour at 4° C. Unbound antibodies were removed by washing two times with PBS+2% FBS. The cells were incubated with 1 μg/mL of fluorescein isothiocyanate (FITC) labeled Goat F(ab′)2 Anti-Mouse IgG secondary antibody (Southern Biotech 1032-02) for 45 min. at 4° C. After washing two times with PBS+2% FBS, cells were resuspended in 0.5 μg/mL Propidium Iodide solution (Invitrogen P3566) and loaded onto a BD-FACS Caliber™ machine for sorting.) flow cytometry gating on a healthy cell population.

Fluorescence emission intensity (x-axis on the plots below; each point represents data from a single cell) was greater on average from 293 cells expressing hNav1.7 (FIG. 20A) than from parental HEK 293 cells (FIG. 20B). The interpretation is that the knockout mouse made mouse antibodies, and that these antibodies were directed against hNa_{V}1.7. (Y-axis does not reflect a labeled marker.)

1. An in vivo method of screening candidate test compounds in a mammal for a pain-relieving effect, said method comprising the steps of:
(a) dosing a mammal with a test compound, followed by
(b) dosing the mammal with a dose of a Na_{V}1.7 activator selected from veratridine, deltamethrin, and grayanotoxin III, effective to induce a Na_{V}1.7-specific biochemical challenge producing a pain-associated response in a negative control but not in a global Nav1.7 knockout mammal of the same species that exhibits a phenotype characterized by a lack of thermal pain sensation and presence of anosmia; and then
(c) determining whether the pain-associated response in the mammal is reduced compared to the negative control.

2. (canceled)

3. The method of claim 1, wherein the mammal is a mouse, rat, rabbit, ferret, dog, non-human primate, or human.

4. The method of claim 1, wherein the pain-associated response is paw lifting, paw licking, flinching, vocalization, self-reporting, or a combination of any of these responses.

5. An in vivo method of screening candidate test compounds in a mammal for a pain-relieving effect, said method comprising the steps of:
(a) dosing a first mammal at a first dose of a test compound, followed by
(b) dosing the first mammal with a dose of a Na_{V}1.7 activator selected from veratridine, deltamethrin, and grayanotoxin III, effective to induce a Na_{V}1.7-specific biochemical challenge producing a pain-associated response in a negative control but not in a global Nav1.7 knockout mammal of the same species that exhibits a phenotype characterized by a lack of thermal pain sensation and presence of anosmia; and then
(c) determining whether the pain-associated response is reduced in the first mammal compared to the negative control; and
(d) identifying a lowest second dose of the test compound in the first mammal at which the pain-associated response is reduced compared to the negative control.

6. (canceled)

7. The method of claim 5, wherein the mammal is a mouse, rat, rabbit, ferret, dog, non-human primate, or human.

8. The method of claim 5, wherein the pain-associated response is paw lifting, paw licking, flinching, vocalization, self-reporting, or a combination of any of these responses.

9. The method of claim 5, further comprising:
(e) dosing a second mammal of the same species at the second dose of the test compound, followed by dosing the second mammal with the dose of the Na_{V}1.7 activator in (b); and then
(f) determining whether the pain-associated response is reduced in the second mammal compared to the negative control.

10. An in vivo method of screening candidate test compounds in a mammal for a pain-relieving effect, said method comprising the steps of:
(a) dosing a first mammal at a first dose of a test compound and a second mammal of the same species at a second dose of the test compound different from the first dose, followed by
(b) dosing the first and the second mammals with a local dose of a Na_{V}1.7 activator selected from veratridine, deltamethrin, and grayanotoxin III, effective to induce a Na_{V}1.7-specific biochemical challenge producing a pain-associated response in a negative control but not in a global Nav1.7 knockout mammal of the same species that exhibits a phenotype characterized by a lack of thermal pain sensation and presence of anosmia; and then
(c) determining whether the pain-associated response is reduced in the first mammal and the second mammal compared to the negative control; and
(d) identifying a lowest second dose of the test compound in the first and the second mammals at which the pain-associated response is reduced compared to the negative control.

11. (canceled)

12. The method of claim 10, wherein the mammal is a mouse, rat, rabbit, ferret, dog, non-human primate, or human.

13. The method of claim 10, wherein the pain-associated response is paw lifting, paw licking, flinching, vocalization, self-reporting, or a combination of any of these responses.
